(19) United States
(12) Patent Application Publication
(10) Pub. No.: US 2016/0017360 A1
(43) Pub. Date: Jan. 21, 2016

(54) FUNCTIONAL EXPRESSION OF BACTERIAL MAJOR FACILITATOR SUPERFAMILY MFS GENE IN MAIZE TO IMPROVE AGRONOMIC TRAITS AND GRAIN YIELD

(71) Applicants: PIONEER HI-BRED INTERNATIONAL, INC., Johnston, IA (US); E.I. DUPONT DE NEMOURS & COMPANY, Wilmington, DE (US)

(72) Inventors: Robel KAHSAY, NEWARK, DE (US); Dale F. LOUSSAERT, CLIVE, IA (US); Bo SHEN, JOHNSTON, IA (US); Jean-Francois TOMB, WILMINGTON, DE (US); Haiyin WANG, JOHNSTON, IA (US); Zhenglin HOU, ANKENY, IA (US)

(21) Appl. No.: 14/770,905

(22) PCT Filed: Mar. 5, 2014
(86) PCT No.: PCT/US14/20642

§ 371 Date: Aug. 27, 2015

Related U.S. Application Data
(60) Provisional application No. 61/779,339, filed on Mar. 13, 2013.

(51) Int. Cl. C12N 15/82

(52) U.S. Cl. CPC C12N 15/8261; C12N 15/8273

(57) ABSTRACT

Methods for modulating plants using optimized Bacterial MFS constructs are disclosed. Also disclosed are nucleotide sequences, constructs, vectors, and modified plant cells, as well as transgenic plants displaying increased seed and/or biomass yield, improved tolerance to abiotic stress such as drought or high plant density, improved nitrogen utilization efficiency, increased ear tissue growth or kernel number.

### FIELD

This disclosure relates generally to the field of molecular biology and the modulation of expression or activity of genes and proteins affecting yield, abiotic stress tolerance and nitrogen utilization efficiency in plants.

### BACKGROUND

Grain yield improvements by conventional breeding have nearly reached a plateau in maize. It is natural then to explore some alternative, non-conventional approaches that could be employed to obtain further yield increases. However, to meet the demand of rapid population in future, much more increases in food production is required. The scale of the increase requires the involvement of new technologies such as transgene-based improvement in agronomic traits. The disclosure can be used for transgene-based improvements of agronomic traits. The described gene can be used to improve N use efficiency, increase grain yield and shorten crop maturity.

Nitrate is the major nitrogen source for maize. Nitrate uptake is an active process which is against an electrochemical potential gradient of the plasma membranes and facilitated by nitrate transporters. Nitrate transporters are also involved in nitrate translocation within the plants. Nitrate transporters are classified into low- and high-affinity nitrate transporter systems (LATS and HATS). Two-component HATS composed of a typical carrier-type protein (NRT2) and an additional small associated membrane protein (NAR2) are reported in green algae and plants and single-component HATS are mostly found in bacteria, fungi, and algae. LATS is a typical carrier-type protein containing ˜12 transmembrane. NRT2 and NRT1 share less homolog in sequences and belong to Major Facilitator Superfamily (MFS) and Peptide Transporter (PTR) family, respectively. In general, LATS are active when soil nitrate concentration is higher than 1 mM and HATS are responsible when soil nitrate concentration is lower than 1 mM.

A transgenic approach via overexpressing single-component HATS to improve nitrate uptake and yield is promosing. Yeast nitrate transporter (YNT1) and YNT1 shuffled variants showed yield advantage in the field. To expand the diversity of canadiate genes to be tested in transgenic maize pipeline, bacterial MFS genes were inditified with a blast search against the GenBank non-redundant database (NR) and environmental database ENV_NR as well as GQPAT (patent database from GenomeQuest). After studying on thousands of putative bacterial MFS sequences, 52 MFSs were nominated from 33 non-pathogenic bacterial strains. MFSs were selected, codon optimized for maize expression, driven by root-preferred promoter (ZmRM2 PRO) and/or constitutive promoter (ZmUBI PRO), and transformed into GS3xGaspe background. Constructs had efficacy to improve ear related traits, e.g. ear length, ear width, ear area, and/or seed numbers under 4 mM nitrate conditions at T0 generation and advanced to further phenotyping on ear traits and nitrate uptake at T1 generation. The constructs had efficacy to improve ear related traits at T1 reproduction assay under limited either nitrogen or water conditions were transformed into elite background for yield testing in field. The MFSs have potential to be used to develop commercial products which improve NUE and other agronomic traits, including further optimization and/or stacking with other genes for improving abiotic stress tolerance, nitrate assimilations and/or root structures. This disclosure also demonstrates that LATS is plant-specific after the blast search.

### SUMMARY

Nitrate uptake is an active process and facilitated by nitrate transporters. The two components (NRT2 and NAR2) of plant HATS interact directly and are required to uptake nitrate. Over-expression of tobacco endogenous high affinity nitrate transporters (NRT2) failed to improve nitrate uptake due to missing the associated protein (Fraisier, et al., 2000). Plant HATS also involved in nitrate response signaling and regulation of root growth. Modification of endogenous Nrt2 expression may cause negative effects. However, over-expression of a single-component high affinity nitrate transporter from non-plant organisms, e.g. yeast or bacteria, will overcome these disadvantages and improve nitrate utilization efficiency.

Seventeen gene families of transporters were classified based on sequence similaririties within MFS (Pao, et al., 1998) and 15 gene families contain transporter genes from bacteria which include transporters for sugar, oligosaccharide, metabolite, nitrate, phosphate, nucleoside, anion and/or drugs. However, the substrate specificity of a given MFS gene needs to be experimental confirmed.

The individual bacterial MFS was expressed in maize to improve nitrate uptake and/or grain yield. Transgenic plants with Bacterial MFS expression, produced statistically significant increase of ear length, ear width, ear area and/or kernel number per ear in T0 phenomics in GS3/GASPE/GASPE background. Therefore, the growth condition also affects the plants productivity. However, in both environments the transgenic plants produced longer ears than the non transgenic control plants. The constructs had efficacy to improve ear related traits at T0 were advanced to T1 reproductive assay under either limited nitrogen source, e.g. 4 mM nitrate or limited water conditions, e.g., 25% of water application for further ear related trait evaluation. Ear growth is reduced in maize under stressed environments, such as drought and low nitrogen stress or nutrient deficiency, which ultimately contribute to grain yield reduction. The prolificacy of the Bacterial MFS transgenic plants offers opportunities to improve yield under the stressed growth environments.

There is a continuing need for modulation of remobilization in plants for manipulating plant development or biomass. This disclosure relates to the creation of Bacterial MFS polynucleotide constructs to modulate yield as seed and/or biomass, abiotic stress tolerance, including density tolerance, drought tolerance, low nitrogen stress, nitrogen utilization efficiency and/or other modifications in plants, including polynucleotide sequences, expression cassettes, constructs, vectors, plant cells and resultant plants. These and other features of the disclosure will become apparent upon review of the following.

This disclosure provides methods and compositions for modulating yield, drought tolerance, low nitrogen stress and/or nitrogen utilization efficiency in plants as well as speeding up remobilization of nutrients including nitrogen in plants. This disclosure relates to compositions and methods for modulating the level and/or activity of Bacterial MFS in plants, exemplified by, e.g., SEQ ID 4: 186470958 from Burkholderia phymatum STM815, SEQ ID 19: 220921692 from Methylobacterium nodulans ORS 2060, SEQ ID 35: 228947460 from Bacillus thuringiensis serovar monterrey BGSC 4AJ1 and SEQ ID 38: 229538083 from Planctomyces limnophilus DSM 3776, for creation of plants with improved yield and/or improved abiotic stress tolerance, which may include improved drought tolerance, improved density tolerance, enhanced yield or nitrogen (fertilizer) response in yield under high nitrogen (current commercial hybrids level off of the yield at high fertilizer application) and/or improved NUE (nitrogen utilization efficiency). NUE includes both improved yield in low nitrogen conditions and more efficient nitrogen utilization in normal conditions.

Therefore, in one aspect, the present disclosure relates to an isolated nucleic acid comprising a polynucleotide sequence which modulates Bacterial MFS expression. One embodiment of the disclosure is an isolated polynucleotide comprising a nucleotide sequence of SEQ ID NO: 4, 19, 35 or 38.

In another aspect, the present disclosure relates to recombinant constructs comprising the polynucleotides as described (see, SEQ ID NO: 4, 19, 35 and 38). The constructs generally comprise the polynucleotides of SEQ ID NO: 4, 19, 35 or SEQ ID NO: 38 and a promoter operably linked to the same. Additionally, the constructs include several features which facilitate modulation of Bacterial MFS expression. The disclosure also relates to a vector containing the recombinant expression cassette. Further, the vector containing the recombinant expression cassette can facilitate the transcription of the nucleic acid in a host cell. The present disclosure also relates to the host cells able to transcribe a polynucleotide.

In certain embodiments, the present disclosure is directed to a transgenic plant or plant cell containing a polynucleotide comprising the construct. In certain embodiments, a plant cell of the disclosure is from a dicot or monocot. Preferred plants containing the polynucleotides include, but are not limited to, maize, soybean, sunflower, sorghum, canola, wheat, alfalfa, cotton, rice, barley, tomato and millet. In certain embodiments, the transgenic plant is a maize plant or plant cell. A transgenic seed comprising a transgenic construct as described herein is an embodiment. In one embodiment, the plant cell is in a hybrid plant comprising a drought tolerance phenotype and/or a nitrogen utilization efficiency phenotype and/or an improved yield phenotype. In another embodiment, the plant cell is in a plant comprising a sterility phenotype, e.g., a male sterility phenotype. Plants may comprise a combination of such phenotypes. A plant regenerated from a plant cell of the disclosure is also a feature of the disclosure.

Certain embodiments have improved drought tolerance as compared to a control plant. The improved drought tolerance of a plant of the disclosure may reflect physiological aspects such as, but not limited to, (a) an increase in the production of at least one MFS-encoding polynucleotide; (b) an increase in the production of a Bacterial MFS polypeptide; (c) changes in ear tissue development rate; (d) an increase in sink capacity; (e) an increase in plant tissue growth or (f) any combination of (a)-(e), compared to a corresponding control plant. Plants exhibiting improved drought tolerance may also exhibit one or more additional abiotic stress tolerance phenotyopes, such as improved nitrogen utilization efficiency and increased density tolerance.

The disclosure also provides methods using nitrate transporter expression for increasing yield component expression in a plant and plants produced by such methods. For example, a method of increasing Bacterial MFS production comprises increasing the expression of one or more Bacterial MFS genes in the plant, wherein the one or more Bacterial MFS genes encode one or more transporter. Multiple methods and/or multiple constructs may be used to increase a single transporter polynucleotide or polypeptide. Multiple Bacterial MFS polynucleotides or polypeptides may be increased in a plant by a single method or by multiple methods; in either case, one or more compositions may be employed.

Methods for modulating drought tolerance in plants are also a feature of the disclosure, as are plants produced by such methods. For example, a method of modulating drought tolerance comprises: (a) selecting at least one Bacterial MFS gene to impact, thereby providing at least one desired Bacterial MFS gene; (b) introducing a mutant form of the at least one desired Bacterial MFS gene into the plant and (c) expressing the mutant form, thereby modulating drought tolerance in the plant. In certain embodiments, the mutant gene is introduced by Agrobacterium-mediated transfer, electroporation, micro-projectile bombardment, a sexual cross or the like.

Detection of expression products is performed either qualitatively (by detecting presence or absence of one or more product of interest) or quantitatively (by monitoring the level of expression of one or more product of interest). In one embodiment, the expression product is an RNA expression product. Aspects of the disclosure optionally include monitoring an expression level of a nucleic acid, polypeptide or chemical, seed production, senesence, dry down rate, etc., in a plant or in a population of plants.

Kits which incorporate one or more of the nucleic acids noted above are also a feature of the disclosure. Such kits can include any of the above noted components and further include, e.g., instructions for use of the components in any of the methods noted herein, packaging materials and/or containers for holding the components. For example, a kit for detection of Bacterial MFS expression levels in a plant includes at least one polynucleotide sequence comprising a nucleic acid sequence, where the nucleic acid sequence is, e.g., at least about 70%, at least about 75%, at least about 80%, at least about 85%, at least about 90%, at least about 95%, at least about 99%, about 99.5% or more, identical to SEQ ID NO: 4, 19, 35, or 38 or a subsequence thereof or a complement thereof. The subsequence may be SEQ ID NO: 127-135 or 121-126 or 105-106. In a further embodiment, the kit includes instructional materials for the use of the at least one polynucleotide sequence to modulate drought tolerance in a plant.

### BRIEF DESCRIPTION OF THE DRAWINGS

FIG. 1: Dendrogram illustrating the relationship among the 52 MFS polypeptides of this disclosure from various bacterial species.

FIG. 2 (as FIG. 2a-FIG. 2q) Sequence alignment of 52 MFS polypeptides from various bacterial species.

FIG. 3: Effects of transgenic NRT2.2 (BP) (SEQ ID: 4) on plant ear related traits under 75% water reduction at T1 generation. Three events of PHP50688 (ZmRM2:ADHI Intron:NRT2.2 (BP) with 1-2 copy of transgene were selected for T1 water use efficiency (WUE) reproductive assay under limited water application. The following traits were measured: ear area (cm²), ear length (cm), ear width (cm), and silk count. Trangenic positive plants tend to have increased ear area, ear length, ear width, and/or silk numbers compared to non-transgenic nulls. Asterisks indicate significance at p<0.1.

FIG. 4 (as FIG. 4a-FIG. 4d) Effects of transgenic plants expressing bacterial MFS genes on plant ear related traits under 4 mM nitrate conditions at T1 generation. T1 nitrogen use efficiency (NUE) reproductive assay was conducted on transgenic plants carrying PHP50688 (ZmRM2:ADHI Intron:NRT2.2 (BP) (FIG. 4a), PHP50692 (ZmRM2:ADHI Intron:NRT2.1 (MN) (FIG. 4b), PHP50693 (ZmRM2:ADHI Intron:NRT2.1 (BP) (FIG. 4c), or PHP50697 (ZmRM2:ADHI Intron:NRT2.1 (PL) (FIG. 4d). Three events with 1-2 copy of transgene from each construct were selected for T1 reproductive assay under limited nitrate application (4 mM nitrate). The following traits were measured: ear area (cm²), ear length (cm), ear width (cm), and silk count. Trangenic positive plants tend to have increased ear area, ear length, ear width, and/or silk numbers compared to non-transenic nulls. Asterisks indicate significance at p<0.1.

FIG. 5: Dendrogram illustrating the clade Genus/Family of the 6 MFS polypeptides showed efficacy on ear traits at T0 generation.

FIG. 6 (as FIG. 6a-FIG. 6p): Sequence alignment of 31 identified MFS polypeptides from 6 clades Genus/Family.

FIG. 7: The cartoon representation of NarK (PDB:4iu8). Left panel (FIG. 7a) is a view from cytoplasm while right panel (FIG. 7b) is a view in membrane plane. Bound NO3 is represented by a 4 ball shaped model in left panel, while the key binding site residues are showed with arrow in right panel.

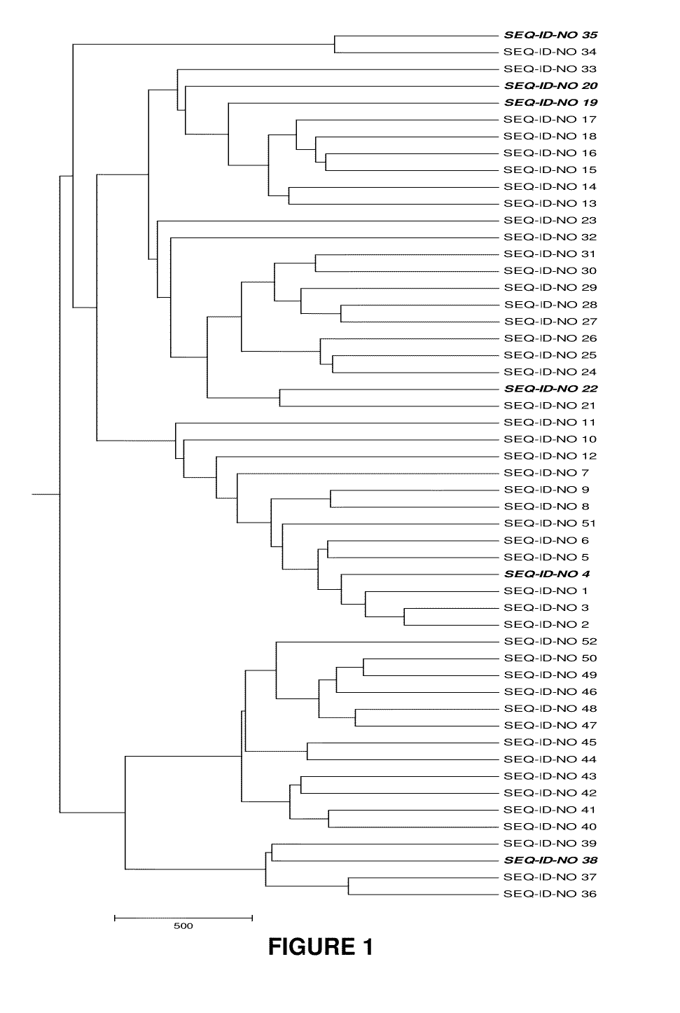

### DETAILED DESCRIPTION

It is to be understood that the terminology used herein is for the purpose of describing particular embodiments only and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

Unless described otherwise, all technical and scientific terms used herein have the same meaning as commonly understood by one of ordinary skill in the art to which the disclosure pertains. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

The present disclosures now will be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5th ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984) and the series Methods in Enzymology, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, DC (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);
2) Aspartic acid (D), Glutamic acid (E);
3) Asparagine (N), Glutamine (Q);

4) Arginine (R), Lysine (K);
5) Isoleucine (I), Leucine (L), Methionine (M), Valine (V); and
6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “nitrate uptake-associated nucleic acid” means a nucleic acid comprising a polynucleotide (“nitrate uptake-associated polynucleotide”) encoding a full length or partial length nitrate uptake-associated polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example) and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions.

The term “nitrate uptake-associated polypeptide” refers to one or more amino acid sequences. The term is also inclusive of fragments, variants, homologs, alleles or precursors (e.g., preproproteins or proproteins) thereof. A “nitrate uptake-associated protein” comprises a nitrate uptake-associated polypeptide. Unless otherwise stated, the term “nitrate uptake-associated nucleic acid” means a nucleic acid comprising a polynucleotide (“nitrate uptake-associated polynucleotide”) encoding a nitrate uptake-associated polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene.

The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The disclosure discloses nitrate uptake-associated polynucleotides and polypeptides. The nucleotides and proteins of the disclosure have an expression pattern which indicates that they enhance nitrogen uptake and utilization and thus play an important role in plant development. The polynucleotides are expressed in various plant tissues. The polynucleotides and polypeptides thus provide an opportunity to manipulate plant development to alter tissue development, timing or composition. This may be used to create a plant with enhanced yield under limited nitrogen supply.

Nucleic Acids

The present disclosure provides, inter alia, isolated nucleic acids of RNA, DNA, homologs, paralogs and orthologs and/or chimeras thereof, comprising a nitrate uptake-associated polynucleotide. This includes naturally occurring as well as synthetic variants and homologs of the sequences.

Sequences homologous, i.e., that share significant sequence identity or similarity, to those provided herein derived from maize, Arabidopsis thaliana or from other plants of choice, are also an aspect of the disclosure. Homologous sequences can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf or fruits and vegetables, such as banana, blackberry, blueberry, strawberry and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassaya, turnip, radish, yam and sweet potato and beans. The homologous sequences may also be derived from woody species, such pine, poplar and eucalyptus or mint or other labiates. In addition, homologous sequences may be derived from plants that are evolutionarily-related to crop plants, but which may not have yet been used as crop plants. Examples include deadly nightshade (Atropa belladona), related to tomato; jimson weed (Datura strommium), related to peyote; and teosinte (Zea species), related to corn (maize).

Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson, et al., (1994) Nucleic Acids Res. 22:4673-4680; Higgins, et al., (1996) Methods Enzymol. 266:383-402). Groups of similar genes can also be identified with pair-wise BLAST analysis (Feng and Doolittle, (1987) J. Mol. Evol. 25:351-360).

For example, a clade of very similar MADS domain transcription factors from Arabidopsis all share a common function in flowering time (Ratcliffe, et al., (2001) Plant Physiol. 126:122-132) and a group of very similar AP2 domain transcription factors from Arabidopsis are involved in tolerance of plants to freezing (Gilmour, et al., (1998) Plant J. 16:433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences or orthologous sequences that share the same function (see also, for example, Mount, (2001) in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson, et al., (1994) Nucleic Acids Res. 22:4673-4680; Higgins, et al., (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms have highly conserved functions and very often essentially identical functions (Lee, et al., (2002) Genome Res. 12:493-502; Remm, et al., (2001) J. Mol. Biol. 314:1041-1052). Paralogous genes, which have diverged through gene duplication, may retain similar functions of the encoded proteins. In such cases, paralogs can be used interchangeably with respect to certain embodiments of the instant disclosure (for example, transgenic expression of a coding sequence).

Variant Nucleotide Sequences in the Non-Coding Regions

The nitrate uptake-associated nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO: 1. These variants are then associated with natural variation in the germplasm for component traits related to NUE. The associated variants are used as marker haplotypes to select for the desirable traits.

Variant Amino Acid Sequences of Nitrate Uptake-Associated Polypeptides

Variant amino acid sequences of the Nitrate uptake associated polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined herein is followed. Variants having about 70%, 75%, 80%, 85%, 90% and 95% nucleic acid sequence identity are generated using this method. These variants are then associated with natural variation in the germplasm for component traits related to NUE. The associated variants are used as marker haplotypes to select for the desirable traits.

The present disclosure also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 4 of Murray, et al., supra.

The nitrate uptake-associated nucleic acids of the present disclosure comprise isolated nitrate uptake-associated polynucleotides which are inclusive of:

(a) a polynucleotide encoding a nitrate uptake-associated polypeptide and conservatively modified and polymorphic variants thereof;

(b) a polynucleotide having at least 70% sequence identity with polynucleotides of (a) or (b);

(c) complementary sequences of polynucleotides of (a) or (b).

The following table, Table 1, lists the specific identities of disclosed polypeptides.

TABLE 1

| Source ID | Bacterial strain | Name | SEQ ID NO: |
|---|---|---|---|
| 94311011 | Cupriavidus metallidurans CH34 | NRT2.1 (CM) | SEQ ID NO: 1 |
| 189423282 | Geobacter lovleyi SZ | NRT2.1 (GL) | SEQ ID NO: 2 |
| 189425713 | Geobacter lovleyi SZ | NRT2.2 (GL) | SEQ ID NO: 3 |
| 186470958 | Burkholderia phymatum STM815 | NRT2.2 (BP) | SEQ ID NO: 4 |
| 110679686 | Roseobacter denitrificans OCh | NRT2.1 (RD) | SEQ ID NO: 5 |
| | 114 | | |
| 167647824 | Caulobacter sp. K31 | NRT2.1 (C-K31) | SEQ ID NO: 6 |
| 25188166 | Selenomonas ruminantium | NRT2.1 (SR) | SEQ ID NO: 7 |
| 283846259 | Bacillus cellulosilyticus DSM 2522 | NRT2.1 (BC) | SEQ ID NO: 8 |
| 283845245 | Bacillus cellulosilyticus DSM 2522 | NRT2.2 (BC) | SEQ ID NO: 9 |
| 18413617 | Halomonas halodenitrificans | NRT2.2 (HH) | SEQ ID NO: 10 |
| 25027853 | Corynebacterium efficiens YS-314 | NRT2.1 (CE) | SEQ ID NO: 11 |
| 145596376 | Salinispora tropica CNB-440 | NRT2.1 (ST) | SEQ ID NO: 12 |
| 52078816 | Bacillus licheniformis ATCC | NRT2.1 (BL) | SEQ ID NO: 13 |
| | 14580 | | |
| 261406348 | Geobacillus sp. Y412MC10 | NRT2.1 (G- | SEQ ID NO: 14 |
| | | Y412MC10) | |
| 167646919 | Caulobacter sp. K31 | NRT2.2 (C-K31) | SEQ ID NO: 15 |
| 119897579 | Azoarcus sp. BH72 | NRT2.1 (A-BH72) | SEQ ID NO: 16 |
| 188991700 | Xanthomonas campestris pv. | NRT2.1 (XC) | SEQ ID NO: 17 |
| | campestris str. B100 | | |
| 146282747 | Pseudomonas stutzeri A1501 | NRT2.1 (PS) | SEQ ID NO: 18 |
| 220921692 | Methylobacterium nodulans ORS | NRT2.1 (MN) | SEQ ID NO: 19 |
| | 2060 | | |
| 186477088 | Burkholderia phymatum STM815 | NRT2.1 (BP) | SEQ ID NO: 20 |
| 145592742 | Salinispora tropica CNB-440 | NRT2.1 (ST) | SEQ ID NO: 21 |
| 25027972 | Corynebacterium efficiens YS-314 | NRT2.2 (CE) | SEQ ID NO: 22 |
| 189423281 | Geobacter lovleyi SZ | NRT2.3 (GL) | SEQ ID NO: 23 |
| 74317419 | Thiobacillus denitrificans ATCC | NRT2.1 (TD) | SEQ ID NO: 24 |
| | 25259 | | |
| 94311010 | Cupriavidus metallidurans CH34 | NRT2.2 (CM) | SEQ ID NO: 25 |
| 189425712 | Geobacter lovleyi SZ | NRT2.4 (GL) | SEQ ID NO: 26 |
| 220922117 | Methylobacterium nodulans ORS | NRT2.2 (MN) | SEQ ID NO: 27 |
| | 2060 | | |
| 167647823 | Caulobacter sp. K31 | NRT2.3 (C-K31) | SEQ ID NO: 28 |
| 18413616 | Halomonas halodenitrificans | NRT2.3 (HH) | SEQ ID NO: 29 |
| 16264170 | Sinorhizobium meliloti 1021 | NRT2.1 (SM) | SEQ ID NO: 30 |
| 119387158 | Paracoccus denitrificans PD1222 | NRT2.1 (PD) | SEQ ID NO: 31 |
| 18413620 | Halomonas halodenitrificans | NRT2.1 (HH) | SEQ ID NO: 32 |
| 227514964 | Lactobacillus fermentum ATCC | NRT2.1 (LF) | SEQ ID NO: 33 |
| | 14931 | | |
| 56962496 | Bacillus clausii KSM-K16 | NRT2.1 (BC) | SEQ ID NO: 34 |
| 228947460 | Bacillus thuringiensis serovar | NRT2.1 (BT) | SEQ ID NO: 35 |
| | monterrey BGSC 4AJ1 | | |
| 186470459 | Burkholderia phymatum STM815 | NRT2.2 (BP) | SEQ ID NO: 36 |
| 94313739 | Ralstonia metallidurans CH34 | NRT2.1 (RM) | SEQ ID NO: 37 |
| 229538083 | Planctomyces limnophilus DSM | NRT2.1 (PL) | SEQ ID NO: 38 |
| | 3776 | | |
| 255035828 | Dyadobacter fermentans DSM | NRT2.1 (DF) | SEQ ID NO: 39 |
| | 18053 | | |
| 254424960 | Synechococcus sp. PCC 7335 | NRT2.1 (S- | SEQ ID NO: 40 |
| | | PCC7335) | |
| 116071859 | Synechococcus sp. BL107 | NRT2.1 (S-BL107) | SEQ ID NO: 41 |
| 186681963 | Nostoc punctiforme PCC 73102 | NRT2.1 (NP) | SEQ ID NO: 42 |
| 254422506 | Synechococcus sp. PCC 7335 | NRT2.2 (S- | SEQ ID NO: 43 |
| | | PCC7335) | |
| 110679685 | Roseobacter denitrificans OCh | NRT2.2 (RD) | SEQ ID NO: 44 |
| | 114 | | |
| 283847378 | Bacillus cellulosilyticus DSM 2522 | NRT2.3 (BC) | SEQ ID NO: 45 |
| 170726972 | Shewanella woodyi ATCC 51908 | NRT2.2 (SW) | SEQ ID NO: 46 |
| 254428533 | Alcanivorax sp. DG881 | NRT2.1 (A- | SEQ ID NO: 47 |
| | | DG881) | |
| 254427883 | Alcanivorax sp. DG881 | NRT2.2 (A- | SEQ ID NO: 48 |
| | | DG881) | |
| 91795059 | Shewanella denitrificans OS217 | NRT2.1 (SD) | SEQ ID NO: 49 |
| 170726230 | Shewanella woodyi ATCC 51908 | NRT2.1 (SW) | SEQ ID NO: 50 |
| 89108312 | Escherichia coli W3110 | NRT2.1 (EC) | SEQ ID NO: 51 |
| P37758 | Escherichia coli K12 | NAR U | SEQ ID NO: 52 |

The following table, Table 2, lists the specific identities of disclosed polynucleotides.

TABLE 2

| Source ID | Bacterial strain | Name | SEQ ID NO: |
|---|---|---|---|
| 94311011 | Cupriavidus metallidurans | NRT2.1 (CM) | SEQ ID NO: 53 |
| | CH34 | | |
| 189423282 | Geobacter lovleyi SZ | NRT2.1 (GL) | SEQ ID NO: 54 |
| 189425713 | Geobacter lovleyi SZ | NRT2.2 (GL) | SEQ ID NO: 55 |
| 186470958 | Burkholderia phymatum | NRT2.2 (BP) | SEQ ID NO: 56 |
| | STM815 | | |
| 110679686 | Roseobacter denitrificans OCh | NRT2.1 (RD) | SEQ ID NO: 57 |
| | 114 | | |
| 167647824 | Caulobacter sp. K31 | NRT2.1 (C-K31) | SEQ ID NO: 58 |
| 25188166 | Selenomonas ruminantium | NRT2.1 (SR) | SEQ ID NO: 59 |
| 283846259 | Bacillus cellulosilyticus DSM | NRT2.1 (BC) | SEQ ID NO: 60 |
| | 2522 | | |
| 283845245 | Bacillus cellulosilyticus DSM | NRT2.2 (BC) | SEQ ID NO: 61 |
| | 2522 | | |
| 18413617 | Halomonas halodenitrificans | NRT2.2 (HH) | SEQ ID NO: 62 |
| 25027853 | Corynebacterium efficiens YS-314 | NRT2.1 (CE) | SEQ ID NO: 63 |
| 145596376 | Salinispora tropica CNB-440 | NRT2.1 (ST) | SEQ ID NO: 64 |
| 52078816 | Bacillus licheniformis ATCC | NRT2.1 (BL) | SEQ ID NO: 65 |
| | 14580 | | |
| 261406348 | Geobacillus sp. Y412MC10 | NRT2.1 (G-Y412MC10) | SEQ ID NO: 66 |
| 167646919 | Caulobacter sp. K31 | NRT2.2 (C-K31) | SEQ ID NO: 67 |
| 119897579 | Azoarcus sp. BH72 | NRT2.1 (A-BH72) | SEQ ID NO: 68 |
| 188991700 | Xanthomonas campestris pv. | NRT2.1 (XC) | SEQ ID NO: 69 |
| | campestris str. B100 | | |
| 146282747 | Pseudomonas stutzeri A1501 | NRT2.1 (PS) | SEQ ID NO: 70 |
| 220921692 | Methylobacterium nodulans | NRT2.1 (MN) | SEQ ID NO: 71 |
| | ORS 2060 | | |
| 186477088 | Burkholderia phymatum | NRT2.1 (BP) | SEQ ID NO: 72 |
| | STM815 | | |
| 145592742 | Salinispora tropica CNB-440 | NRT2.1 (ST) | SEQ ID NO: 73 |
| 25027972 | Corynebacterium efficiens YS- | NRT2.2 (CE) | SEQ ID NO: 74 |
| | 314 | | |
| 189423281 | Geobacter lovleyi SZ | NRT2.3 (GL) | SEQ ID NO: 75 |
| 74317419 | Thiobacillus denitrificans ATCC | NRT2.1 (TD) | SEQ ID NO: 76 |
| | 25259 | | |
| 94311010 | Cupriavidus metallidurans | NRT2.2 (CM) | SEQ ID NO: 77 |
| | CH34 | | |
| 189425712 | Geobacter lovleyi SZ | NRT2.4 (GL) | SEQ ID NO: 78 |
| 220922117 | Methylobacterium nodulans | NRT2.2 (MN) | SEQ ID NO: 79 |
| | ORS 2060 | | |
| 167647823 | Caulobacter sp. K31 | NRT2.3 (C-K31) | SEQ ID NO: 80 |
| 18413616 | Halomonas halodenitrificans | NRT2.3 (HH) | SEQ ID NO: 81 |
| 16264170 | Sinorhizobium meliloti 1021 | NRT2.1 (SM) | SEQ ID NO: 82 |
| 119387158 | Paracoccus denitrificans | NRT2.1 (PD) | SEQ ID NO: 83 |
| | PD1222 | | |
| 18413620 | Halomonas halodenitrificans | NRT2.1 (HH) | SEQ ID NO: 84 |
| 227514964 | Lactobacillus fermentum ATCC | NRT2.1 (LF) | SEQ ID NO: 85 |
| | 14931 | | |
| 56962496 | Bacillus clausii KSM-K16 | NRT2.1 (BC) | SEQ ID NO: 86 |
| 228947460 | Bacillus thuringiensis serovar | NRT2.1 (BT) | SEQ ID NO: 87 |
| | monterrey BGSC 4AJ1 | | |
| 186470459 | Burkholderia phymatum | NRT2.2 (BP) | SEQ ID NO: 88 |
| | STM815 | | |
| 94313739 | Ralstonia metallidurans CH34 | NRT2.1 (RM) | SEQ ID NO: 89 |
| 229538083 | Planctomyces limnophilus DSM | NRT2.1 (PL) | SEQ ID NO: 90 |
| | 3776 | | |
| 255035828 | Dyadobacter fermentans DSM | NRT2.1 (DF) | SEQ ID NO: 91 |
| | 18053 | | |
| 254424960 | Synechococcus sp. PCC 7335 | NRT2.1 (S-PCC7335) | SEQ ID NO: 92 |
| 116071859 | Synechococcus sp. BL107 | NRT2.1 (S-BL107) | SEQ ID NO: 93 |
| 186681963 | Nostoc punctiforme PCC 73102 | NRT2.1 (NP) | SEQ ID NO: 94 |
| 254422506 | Synechococcus sp. PCC 7335 | NRT2.2 (S-PCC7335) | SEQ ID NO: 95 |
| 110679685 | Roseobacter denitrificans OCh | NRT2.2 (RD) | SEQ ID NO: 96 |
| | 114 | | |
| 283847378 | Bacillus cellulosilyticus DSM | NRT2.3 (BC) | SEQ ID NO: 97 |
| | 2522 | | |
| 170726972 | Shewanella woodyi ATCC | NRT2.2 (SW) | SEQ ID NO: 98 |
| | 51908 | | |
| 254428533 | Alcanivorax sp. DG881 | NRT2.1 (A-DG881) | SEQ ID NO: 99 |
| 254427883 | Alcanivorax sp. DG881 | NRT2.2 (A-DG881) | SEQ ID NO: |
| | | | 100 |
| 91795059 | Shewanella denitrificans OS217 | NRT2.1 (SD) | SEQ ID NO: |
| | | | 101 |
| 170726230 | Shewanella woodyi ATCC | NRT2.1 (SW) | SEQ ID NO: |
| | 51908 | | 102 |
| 89108312 | Escherichia coli W3110 | NRT2.1 (EC) | SEQ ID NO: |
| | | | 103 |
| P37758 | Escherichia coli K12 | NAR U | SEQ ID NO: |
| | | | 104 |

The following table, Table 3, lists the specific identities of further disclosed polypeptides which belong to the same clade Genus/Family with SEQ ID NO: 4, SEQ ID NO: 19, SEQ ID NO: 20, SEQ ID NO: 22, SEQ ID NO: 35 or SEQ ID NO: 38.

TABLE 3

| Source ID | Baterial strain | SEQ ID NO |
|---|---|---|
| 430746446 | Singulisphaera acidiphila DSM 18658 | 105 |
| 149176108 | Planctomyces maris DSM 8797 | 106 |
| 295676596 | Burkholderia sp. CCGE1002 | 107 |
| 323525977 | Burkholderia sp. CCGE1001 | 108 |
| 187923975 | Burkholderia phytofirmans PsJN | 109 |
| 413963671 | Burkholderia sp. SJ98 | 110 |
| 377819760 | Burkholderia sp. YI23 | 111 |
| 167837863 | Burkholderia thailandensis MSMB43 | 112 |
| 167825807 | Burkholderia pseudomallei 9 | 113 |
| 402570093 | Burkholderia cepacia GG4 | 114 |
| 330815538 | Burkholderia gladioli BSR3 | 115 |
| 167912528 | Burkholderia pseudomallei 112 | 116 |
| 167564088 | Burkholderia oklahomensis EO147 | 117 |
| 53720535 | Burkholderia pseudomallei K96243 | 118 |
| 83720264 | Burkholderia thailandensis E264 | 119 |
| 403520107 | Burkholderia pseudomallei BPC006 | 120 |
| 393766714 | Methylobacterium sp. GXF4 | 121 |
| 170749323 | Methylobacterium radiotolerans JCM 2831 | 122 |
| 46204161 | Magnetospirillum magnetotacticum MS-1 | 123 |
| 163849758 | Methylobacterium extorquens PA1 | 124 |
| 188579650 | Methylobacterium populi BJ001 | 125 |
| 170742934 | Methylobacterium sp. 4-46 | 126 |
| 170692901 | Burkholderia graminis C4D1M | 127 |
| 120609359 | Acidovorax citrulli AAC00-1 | 128 |
| 333916999 | Delftia sp. Cs1-4 | 129 |
| 121592866 | Acidovorax sp. JS42 | 130 |
| 319761229 | Alicycliphilus denitrificans BC | 131 |
| 301631439 | Xenopus (Silurana) tropicalis | 132 |
| 407937190 | Acidovorax sp. KKS102 | 133 |
| 241764574 | Acidovorax delafieldii 2AN | 134 |
| 307726848 | Burkholderia sp. CCGE1003 | 135 |

The following table, Table 4, lists the specific identities of further disclosed polynucleotides which belong to the same clade Genus/Family with SEQ ID NO: 4, SEQ ID NO: 19, SEQ ID NO: 20, SEQ ID NO: 22, SEQ ID NO: 35 or SEQ ID NO: 38.

TABLE 4

| Source ID | Baterial strain | SEQ ID NO |
|---|---|---|
| 430746446 | Singulisphaera acidiphila DSM 18658 | 136 |
| 149176108 | Planctomyces maris DSM 8797 | 137 |
| 295676596 | Burkholderia sp. CCGE1002 | 138 |
| 323525977 | Burkholderia sp. CCGE1001 | 139 |
| 187923975 | Burkholderia phytofirmans PsJN | 140 |
| 413963671 | Burkholderia sp. SJ98 | 141 |
| 377819760 | Burkholderia sp. YI23 | 142 |
| 167837863 | Burkholderia thailandensis MSMB43 | 143 |
| 167825807 | Burkholderia pseudomallei 9 | 144 |
| 402570093 | Burkholderia cepacia GG4 | 145 |
| 330815538 | Burkholderia gladioli BSR3 | 146 |
| 167912528 | Burkholderia pseudomallei 112 | 147 |
| 167564088 | Burkholderia oklahomensis EO147 | 148 |
| 53720535 | Burkholderia pseudomallei K96243 | 149 |
| 83720264 | Burkholderia thailandensis E264 | 150 |
| 403520107 | Burkholderia pseudomallei BPC006 | 151 |
| 393766714 | Methylobacterium sp. GXF4 | 152 |
| 170749323 | Methylobacterium radiotolerans JCM 2831 | 153 |
| 46204161 | Magnetospirillum magnetotacticum MS-1 | 154 |
| 163849758 | Methylobacterium extorquens PA1 | 155 |
| 188579650 | Methylobacterium populi BJ001 | 156 |
| 170742934 | Methylobacterium sp. 4-46 | 157 |
| 170692901 | Burkholderia graminis C4D1M | 158 |
| 120609359 | Acidovorax citrulli AAC00-1 | 159 |
| 333916999 | Delftia sp. Cs1-4 | 160 |
| 121592866 | Acidovorax sp. JS42 | 161 |
| 319761229 | Alicycliphilus denitrificans BC | 162 |
| 301631439 | Xenopus (Silurana) tropicalis | 163 |
| 407937190 | Acidovorax sp. KKS102 | 164 |
| 241764574 | Acidovorax delafieldii 2AN | 165 |
| 307726848 | Burkholderia sp. CCGE1003 | 166 |

The following table, Table 5, lists the specific identities of further disclosed polypeptides having diverse substrate specificity, e.g. sugar transporter.

TABLE 5

| Source ID | Baterial strain | SEQ ID NO |
|---|---|---|
| 398381448 | Rhizobium sp. AP16 | 167 |
| 187925681 | Burkholderia phytofirmans PsJN | 168 |
| 443349358 | Bacillus subtilis subsp. inaquosorum KCTC | 169 |
| | 13429 | |
| 238756214 | Yersinia ruckeri ATCC 29473 | 170 |
| 21233541 | Xanthomonas campestris pv. campestris | 171 |
| | str. ATCC 33913 | |
| 162449514 | Sorangium cellulosum So ce56 | 172 |
| 91779392 | Burkholderia xenovorans LB400 | 173 |
| 322699061 | Metarhizium acridum CQMa 102 | 174 |
| 255556942 | Ricinus communis | 175 |
| 403674253 | Acinetobacter sp. NCTC 10304 | 176 |
| 357413593 | Streptomyces flavogriseus ATCC 33331 | 177 |
| 145222743 | Mycobacterium gilvum PYR-GCK | 178 |
| 403237236 | Bacillus sp. 10403023 | 179 |
| 26988817 | Pseudomonas putida KT2440 | 180 |
| 418051437 | Mycobacterium rhodesiae JS60 | 181 |
| 440695083 | Streptomyces turgidiscabies Car8 | 182 |
| 419763327 | Klebsiella pneumoniae subsp. pneumoniae | 183 |
| | DSM 30104 | |
| 238792474 | Yersinia intermedia ATCC 29909 | 184 |
| 219668545 | Desulfitobacterium hafniense DCB-2 | 185 |
| 134099953 | Saccharopolyspora erythraea NRRL 2338 | 186 |
| 390959594 | Terriglobus roseus DSM 18391 | 187 |
| 399578565 | Halogranum salarium B-1 | 188 |
| 147678099 | Pelotomaculum thermopropionicum SI | 189 |
| 395797926 | Pseudomonas sp. Ag1 | 190 |
| 340347070 | Prevotella dentalis DSM 3688 | 191 |
| 325962868 | Arthrobacter phenanthrenivorans Sphe3 | 192 |
| 381187632 | Flavobacterium frigoris PS1 | 193 |

The following table, Table 6, lists the specific identities of further disclosed polynucleotides having diverse substrate specificity, e.g. sugar transporter.

TABLE 6

| Source ID | Baterial strain | SEQ ID NO |
|---|---|---|
| 398381448 | Rhizobium sp. AP16 | 194 |
| 187925681 | Burkholderia phytofirmans PsJN | 195 |
| 443349358 | Bacillus subtilis subsp. inaquosorum | 196 |
| | KCTC 13429 | |
| 238756214 | Yersinia ruckeri ATCC 29473 | 197 |
| 21233541 | Xanthomonas campestris pv. campestris | 198 |
| | str. ATCC 33913 | |
| 162449514 | Sorangium cellulosum So ce56 | 199 |
| 91779392 | Burkholderia xenovorans LB400 | 200 |
| 322699061 | Metarhizium acridum CQMa 102 | 201 |
| 255556942 | Ricinus communis | 202 |
| 403674253 | Acinetobacter sp. NCTC 10304 | 203 |
| 357413593 | Streptomyces flavogriseus ATCC 33331 | 204 |
| 145222743 | Mycobacterium gilvum PYR-GCK | 205 |
| 403237236 | Bacillus sp. 10403023 | 206 |
| 26988817 | Pseudomonas putida KT2440 | 207 |
| 418051437 | Mycobacterium rhodesiae JS60 | 208 |
| 440695083 | Streptomyces turgidiscabies Car8 | 209 |
| 419763327 | Klebsiella pneumoniae subsp. pneumoniae | 210 |
| | DSM 30104 | |
| 238792474 | Yersinia intermedia ATCC 29909 | 211 |
| 219668545 | Desulfitobacterium hafniense DCB-2 | 212 |
| 134099953 | Saccharopolyspora erythraea NRRL 2338 | 213 |
| 390959594 | Terriglobus roseus DSM 18391 | 214 |
| 399578565 | Halogranum salarium B-1 | 215 |
| 147678099 | Pelotomaculum thermopropionicum SI | 216 |
| 395797926 | Pseudomonas sp. Ag1 | 217 |
| 340347070 | Prevotella dentalis DSM 3688 | 218 |
| 325962868 | Arthrobacter phenanthrenivorans Sphe3 | 219 |
| 381187632 | Flavobacterium frigoris PS1 | 220 |

Construction of Nucleic Acids

The isolated nucleic acids of the present disclosure can be made using (a) standard recombinant methods, (b) synthetic techniques, or combinations thereof. In some embodiments, the polynucleotides of the present disclosure will be cloned, amplified or otherwise constructed from a fungus or bacteria.

The nucleic acids may conveniently comprise sequences in addition to a polynucleotide of the present disclosure. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present disclosure. For example, a hexa-histidine marker sequence provides a convenient means to purify the proteins of the present disclosure. The nucleic acid of the present disclosure—excluding the polynucleotide sequence—is optionally a vector, adapter or linker for cloning and/or expression of a polynucleotide of the present disclosure. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present disclosure less the length of its polynucleotide of the present disclosure is less than 20 kilobase pairs, often less than 15 kb and frequently less than 10 kb. Use of cloning vectors, expression vectors, adapters and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox and lambda MOSElox. Optional vectors for the present disclosure, include but are not limited to, lambda ZAP II, and pGEX. For a description of various nucleic acids see, e.g., Stratagene Cloning Systems, Catalogs 1995, 1996, 1997 (La Jolla, Calif.) and, Amersham Life Sciences, Inc, Catalog '97 (Arlington Heights, Ill.).

Synthetic Methods for Constructing Nucleic Acids

The isolated nucleic acids of the present disclosure can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res.15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

Sequence Shuffling

The present disclosure provides methods for sequence shuffling using polynucleotides of the present disclosure and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters are referred to here as “inducible” promoters. Environmental conditions that may effect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119, and hereby incorporated by reference) or signal peptides which target proteins to the plastids such as that of rapeseed enoyl-Acp reductase (Verwaert, et al., (1994) Plant Mol. Biol. 26:189-202) are useful in the disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al. (1987), Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pB1101.2 that is available from CLONTECH Laboratories, Inc. (Palo Alto, Calif.).

Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

Expression in Eukaryotes

A variety of eukaryotic expression systems such as yeast, insect cell lines, plant and mammalian cells are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al.,(1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the nitrate uptake-associated gene placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

Plant Transformation Methods

Numerous methods for introducing foreign genes into plants are known and can be used to insert a nitrate uptake-associated polynucleotide into a plant host, including biological and physical plant transformation protocols. See, e.g., Miki, et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips. Springer-Verlag Berlin Heidelberg N.Y., 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Cornpositae and Chenopodiaceae. Monocot plants can now be transformed with some success. European Patent Application Number 604 662 A1 discloses a method for transforming monocots using Agrobacterium. European Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S patent application Ser. No. 913,913 and 913,914, both filed Oct. 1, 1986, as referenced in U.S. Pat. No. 5,262,306, issued Nov. 16, 1993, the entire disclosures therein incorporated herein by reference.

Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Int?. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a Nitrate Uptake-Associated Polypeptide

Methods are provided to increase the activity and/or level of the nitrate uptake-associated polypeptide of the disclosure. An increase in the level and/or activity of the nitrate uptake-associated polypeptide of the disclosure can be achieved by providing to the plant a nitrate uptake-associated polypeptide. The nitrate uptake-associated polypeptide can be provided by introducing the amino acid sequence encoding the nitrate uptake-associated polypeptide into the plant, introducing into the plant a nucleotide sequence encoding a nitrate uptake-associated polypeptide or alternatively by modifying a genomic locus encoding the nitrate uptake-associated polypeptide of the disclosure.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having enhanced nitrogen utilization activity. It is also recognized that the methods of the disclosure may employ a polynucleotide that is not capable of directing, in the transformed plant, the expression of a protein or RNA. Thus, the level and/or activity of a nitrate uptake-associated polypeptide may be increased by altering the gene encoding the nitrate uptake-associated polypeptide or its promoter. See, e.g., Kmiec, U.S. Pat. No. 5,565,350; Zarling, et al., PCT/US93/03868. Therefore, mutagenized plants that carry mutations in nitrate uptake-associated genes, where the mutations increase expression of the nitrate uptake-associated gene or increase the nitrate uptake-associated activity of the encoded nitrate uptake-associated polypeptide are provided.

Reducing the Activity and/or Level of a Nitrate Uptake-Associated Polypeptide

Methods are provided to reduce or eliminate the activity of a nitrate uptake-associated polypeptide of the disclosure by transforming a plant cell with an expression cassette that expresses a polynucleotide that inhibits the expression of the nitrate uptake-associated polypeptide. The polynucleotide may inhibit the expression of the nitrate uptake-associated polypeptide directly, by preventing transcription or translation of the nitrate uptake-associated messenger RNA or indirectly, by encoding a polypeptide that inhibits the transcription or translation of a nitrate uptake-associated gene encoding nitrate uptake-associated polypeptide. Methods for inhibiting or eliminating the expression of a gene in a plant are well known in the art, and any such method may be used in the present disclosure to inhibit the expression of nitrate uptake-associated polypeptide. Many methods may be used to reduce or eliminate the activity of a nitrate uptake-associated polypeptide. In addition, more than one method may be used to reduce the activity of a single nitrate uptake-associated polypeptide.

1. Polynucleotide-Based Methods:

In some embodiments of the present disclosure, a plant is transformed with an expression cassette that is capable of expressing a polynucleotide that inhibits the expression of a nitrate uptake-associated polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one nitrate uptake-associated polypeptide is an expression cassette capable of producing an RNA molecule that inhibits the transcription and/or translation of at least one nitrate uptake-associated polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a nitrate uptake-associated polypeptide are given below.

i. Sense Suppression/Cosuppression

In some embodiments of the disclosure, inhibition of the expression of a nitrate uptake-associated polypeptide may be obtained by sense suppression or cosuppression. For cosuppression, an expression cassette is designed to express an RNA molecule corresponding to all or part of a messenger RNA encoding a nitrate uptake-associated polypeptide in the “sense” orientation. Over expression of the RNA molecule can result in reduced expression of the native gene. Accordingly, multiple plant lines transformed with the cosuppression expression cassette are screened to identify those that show the greatest inhibition of nitrate uptake-associated polypeptide expression.

The polynucleotide used for cosuppression may correspond to all or part of the sequence encoding the nitrate uptake-associated polypeptide, all or part of the 5′ and/or 3′ untranslated region of a nitrate uptake-associated polypeptide transcript or all or part of both the coding sequence and the untranslated regions of a transcript encoding a nitrate uptake-associated polypeptide. In some embodiments where the polynucleotide comprises all or part of the coding region for the nitrate uptake-associated polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

ii. Antisense Suppression

In some embodiments of the disclosure, inhibition of the expression of the nitrate uptake-associated polypeptide may be obtained by antisense suppression. For antisense suppression, the expression cassette is designed to express an RNA molecule complementary to all or part of a messenger RNA encoding the nitrate uptake-associated polypeptide. Over expression of the antisense RNA molecule can result in reduced expression of the native gene. Accordingly, multiple plant lines transformed with the antisense suppression expression cassette are screened to identify those that show the greatest inhibition of nitrate uptake-associated polypeptide expression.

The polynucleotide for use in antisense suppression may correspond to all or part of the complement of the sequence encoding the nitrate uptake-associated polypeptide, all or part of the complement of the 5′ and/or 3′ untranslated region of the nitrate uptake-associated transcript or all or part of the complement of both the coding sequence and the untranslated regions of a transcript encoding the nitrate uptake-associated polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a nitrate uptake-associated polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of nitrate uptake-associated polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of a nitrate uptake-associated polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7, and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the nitrate uptake-associated polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the nitrate uptake-associated polypeptide. Thus, the polynucleotide causes the degradation of the endogenous messenger RNA, resulting in reduced expression of the nitrate uptake-associated polypeptide. This method is described, for example, in U.S. Pat. No. 4,987,071, herein incorporated by reference.

vii. Small Interfering RNA or Micro RNA

In some embodiments of the disclosure, inhibition of the expression of a nitrate uptake-associated polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature_{425:257}-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of nitrate uptake-associated expression, the 22-nucleotide sequence is selected from a nitrate uptake-associated transcript sequence and contains 22 nucleotides of said nitrate uptake-associated sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

2. Polypeptide-Based Inhibition of Gene Expression

In one embodiment, the polynucleotide encodes a zinc finger protein that binds to a gene encoding a nitrate uptake-associated polypeptide, resulting in reduced expression of the gene. In particular embodiments, the zinc finger protein binds to a regulatory region of a nitrate uptake-associated gene. In other embodiments, the zinc finger protein binds to a messenger RNA encoding a nitrate uptake-associated polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US. Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one nitrate uptake-associated polypeptide and reduces the enhanced nitrogen utilization activity of the nitrate uptake-associated polypeptide. In another embodiment, the binding of the antibody results in increased turnover of the antibody-nitrate uptake-associated complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of a nitrate uptake-associated polypeptide is reduced or eliminated by disrupting the gene encoding the nitrate uptake-associated polypeptide. The gene encoding the nitrate uptake-associated polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced nitrogen utilization activity.

i. Transposon Tagging

In one embodiment of the disclosure, transposon tagging is used to reduce or eliminate the nitrate uptake-associated activity of one or more nitrate uptake-associated polypeptide. Transposon tagging comprises inserting a transposon within an endogenous nitrate uptake-associated gene to reduce or eliminate expression of the nitrate uptake-associated polypeptide. “nitrate uptake-associated gene” is intended to mean the gene that encodes a nitrate uptake-associated polypeptide according to the disclosure.

In this embodiment, the expression of one or more nitrate uptake-associated polypeptide is reduced or eliminated by inserting a transposon within a regulatory region or coding region of the gene encoding the nitrate uptake-associated polypeptide. A transposon that is within an exon, intron, 5′ or 3′ untranslated sequence, a promoter or any other regulatory sequence of a nitrate uptake-associated gene may be used to reduce or eliminate the expression and/or activity of the encoded nitrate uptake-associated polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function (enhanced nitrogen utilization activity) of the encoded protein are well known in the art. Insertional mutations in gene exons usually result in null-mutants. Mutations in conserved residues are particularly effective in inhibiting the activity of the encoded protein. Conserved residues of plant nitrate uptake-associated polypeptides suitable for mutagenesis with the goal to eliminate nitrate uptake-associated activity have been described. Such mutants can be isolated according to well-known procedures, and mutations in different nitrate uptake-associated loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more nitrate uptake-associated polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Nitrogen Utilization Activity

In specific methods, the level and/or activity of a nitrate uptake-associated regulator in a plant is decreased by increasing the level or activity of the nitrate uptake-associated polypeptide in the plant. The increased expression of a negative regulatory molecule may decrease the level of expression of downstream one or more genes responsible for an improved nitrate uptake-associated phenotype.

Methods for increasing the level and/or activity of nitrate uptake-associated polypeptides in a plant are discussed elsewhere herein.

As discussed above, one of skill will recognize the appropriate promoter to use to modulate the level/activity of a nitrate uptake-associated in the plant. Exemplary promoters for this embodiment have been disclosed elsewhere herein.

In other embodiments, such plants have stably incorporated into their genome a nucleic acid molecule comprising a nitrate uptake-associated nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the nitrate uptake-associated polypeptide in the plant. In one method, a nitrate uptake-associated sequence of the disclosure is provided to the plant. In another method, the nitrate uptake-associated nucleotide sequence is provided by introducing into the plant a polynucleotide comprising a nitrate uptake-associated nucleotide sequence of the disclosure, expressing the nitrate uptake-associated sequence, and thereby modifying root development. In still other methods, the nitrate uptake-associated nucleotide construct introduced into the plant is stably incorporated into the genome of the plant.

In other methods, root development is modulated by altering the level or activity of the nitrate uptake-associated polypeptide in the plant. A change in nitrate uptake-associated activity can result in at least one or more of the following alterations to root development, including, but not limited to, alterations in root biomass and length.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by decreasing the activity and/or level of the nitrate uptake-associated polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by altering the level and/or activity of the nitrate uptake-associated polypeptide also finds use in promoting in vitro propagation of explants.

Furthermore, higher root biomass production due to nitrate uptake-associated activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the nitrate uptake-associated polypeptide of the disclosure and has enhanced root growth and/or root biomass. In other embodiments, such plants have stably incorporated into their genome a nucleic acid molecule comprising a nitrate uptake-associated nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a nitrate uptake-associated polypeptide of the disclosure. In one embodiment, a nitrate uptake-associated sequence of the disclosure is provided. In other embodiments, the nitrate uptake-associated nucleotide sequence can be provided by introducing into the plant a polynucleotide comprising a nitrate uptake-associated nucleotide sequence of the disclosure, expressing the nitrate uptake-associated sequence and thereby modifying shoot and/or leaf development. In other embodiments, the nitrate uptake-associated nucleotide construct introduced into the plant is stably incorporated into the genome of the plant.

In specific embodiments, shoot or leaf development is modulated by altering the level and/or activity of the nitrate uptake-associated polypeptide in the plant. A change in nitrate uptake-associated activity can result in at least one or more of the following alterations in shoot and/or leaf development, including, but not limited to, changes in leaf number, altered leaf surface, altered vasculature, internodes and plant growth and alterations in leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters, and leaf-preferred promoters. Exemplary promoters have been disclosed elsewhere herein.

Increasing nitrate uptake-associated activity and/or level in a plant results in altered internodes and growth. Thus, the methods of the disclosure find use in producing modified plants. In addition, as discussed above, nitrate uptake-associated activity in the plant modulates both root and shoot growth. Thus, the present disclosure further provides methods for altering the root/shoot ratio. Shoot or leaf development can further be modulated by altering the level and/or activity of the nitrate uptake-associated polypeptide in the plant.

Accordingly, the present disclosure further provides plants having modulated shoot and/or leaf development when compared to a control plant. In some embodiments, the plant of the disclosure has an increased level/activity of the nitrate uptake-associated polypeptide of the disclosure. In other embodiments, the plant of the disclosure has a decreased level/activity of the nitrate uptake-associated polypeptide of the disclosure.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the nitrate uptake-associated polypeptide has not been modulated. “Modulating floral development” further includes any alteration in the timing of the development of a plant's reproductive tissue (i.e., a delayed or an accelerated timing of floral development) when compared to a control plant in which the activity or level of the nitrate uptake-associated polypeptide has not been modulated. Macroscopic alterations may include changes in size, shape, number, or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating nitrate uptake-associated activity in a plant. In one method, a nitrate uptake-associated sequence of the disclosure is provided. A nitrate uptake-associated nucleotide sequence can be provided by introducing into the plant a polynucleotide comprising a nitrate uptake-associated nucleotide sequence of the disclosure, expressing the nitrate uptake-associated sequence and thereby modifying floral development. In other embodiments, the nitrate uptake-associated nucleotide construct introduced into the plant is stably incorporated into the genome of the plant.

In specific methods, floral development is modulated by increasing the level or activity of the nitrate uptake-associated polypeptide in the plant. A change in nitrate uptake-associated activity can result in at least one or more of the following alterations in floral development, including, but not limited to, altered flowering, changed number of flowers, modified male sterility and altered seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by altering the level and/or activity of the nitrate uptake-associated sequence of the disclosure. Such methods can comprise introducing a nitrate uptake-associated nucleotide sequence into the plant and changing the activity of the nitrate uptake-associated polypeptide. In other methods, the nitrate uptake-associated nucleotide construct introduced into the plant is stably incorporated into the genome of the plant. Altering expression of the nitrate uptake-associated sequence of the disclosure can modulate floral development during periods of stress. Such methods are described elsewhere herein. Accordingly, the present disclosure further provides plants having modulated floral development when compared to the floral development of a control plant. Compositions include plants having an altered level/activity of the nitrate uptake-associated polypeptide of the disclosure and having an altered floral development. Compositions also include plants having a modified level/activity of the nitrate uptake-associated polypeptide of the disclosure wherein the plant maintains or proceeds through the flowering process in times of stress.

Methods are also provided for the use of the nitrate uptake-associated sequences of the disclosure to increase seed size and/or weight. The method comprises increasing the activity of the nitrate uptake-associated sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for altering seed size and/or seed weight in a plant comprises increasing nitrate uptake-associated activity in the plant. In one embodiment, the nitrate uptake-associated nucleotide sequence can be provided by introducing into the plant a polynucleotide comprising a nitrate uptake-associated nucleotide sequence of the disclosure, expressing the nitrate uptake-associated sequence and thereby increasing seed weight and/or size. In other embodiments, the nitrate uptake-associated nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has a modified level/activity of the nitrate uptake-associated polypeptide of the disclosure and has an increased seed weight and/or seed size. In other embodiments, such plants have stably incorporated into their genome a nucleic acid molecule comprising a nitrate uptake-associated nucleotide sequence of the disclosure operably linked to a promoter that drives expression in the plant cell.

vii. Method of Use for Nitrate Uptake-Associated Polynucleotide, Expression Cassettes, and Additional Polynucleotides

The nucleotides, expression cassettes and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate, or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996 and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502, herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as 13-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

The following examples are offered to illustrate, but not to limit, the claimed subject matter. Various modifications by persons skilled in the art are to be included within the spirit and purview of this application and scope of the appended claims.

### Example 1

### Identification of Bacterial MFS Genes

The identification of bacterial MFS genes was initiated with a blast search against the GenBank non-redundant database (NR) and environmental database ENV_NR as well as GQPAT (patent database from GenomeQuest). 55 query sequences were used in this blast search. Forty five were identified based on known eukaryotic and prokaryotic HATS and LATS proteins and 10 query sequences were extracted from SWISSPROT, the knowledge protein sequence database.

### Details

1. Search for HATS from diverse non-plant organisms.
a. Single component HATS
   i. 4 eukaryotes examples
   ii. 3 bacteria examples
b. Two component HATS
   i. 4 functional examples

2. Identify pure functional LATS.
a. Search for functional LATs
   i. 11 Arabidopsis examples given (10 functional)
   ii. 14 maize examples given (2 functional)
   iii. 4 known PTRs given
b. Dual-affinity nitrate transporter AtNrt1.1. (1 example given)
   i. 1 functional Arabidopsis example given

Protein sequences identified by Blast searches were filtered to include sequences that are within the 400-650 amino acid in length. This is the length range found in the query sequences. Resulting sequences were clustered based on % sequence identity (% ID) and % query length overlap (% L). Clusters at 90%, 70%, 50%, 40% and 30% ID and 70% L were generated. A linkage (member of a cluster) is considered if identity >=% l threshold and query overlap >=70%). Four clusters were identified at the 30% ID and 70% L. One cluster (all HATS sequences) that contained a mix of eukaryotic and prokaryotic sequences was further analyzed. Fifty two candidate sequences were chosen based on phylogeny, cluster information—in the 40% to 90% ID range-, organism physiology and environment. Sequences derived from pathogens or potential pathogens were not included. Three major clusters including PTR/NRT1, MFS/NRT2, and NAR2 gene families were generated. The PTR/NRT1 cluster contains genes from eukaryotic organisms.

### Example 2

### Polypeptide Sequence Analysis

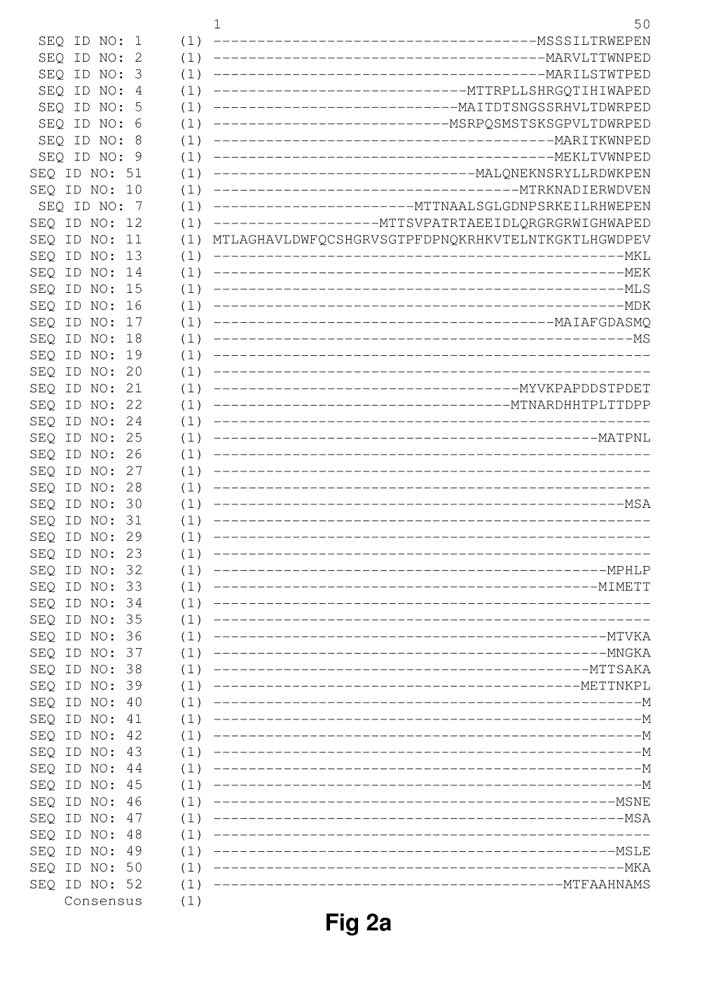

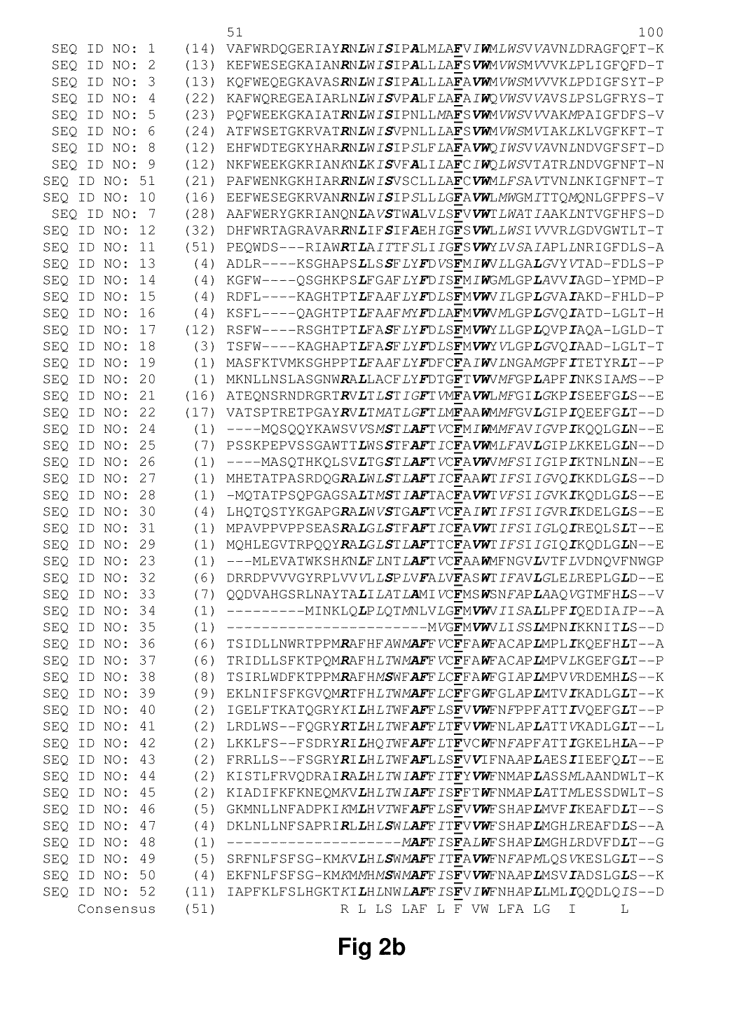

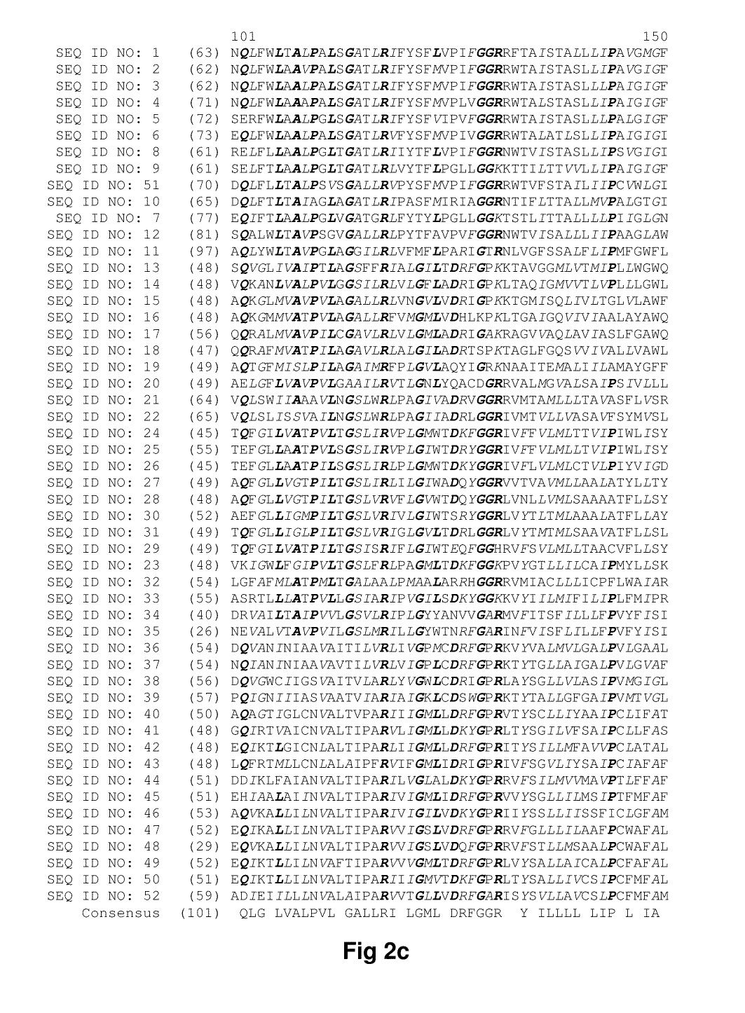

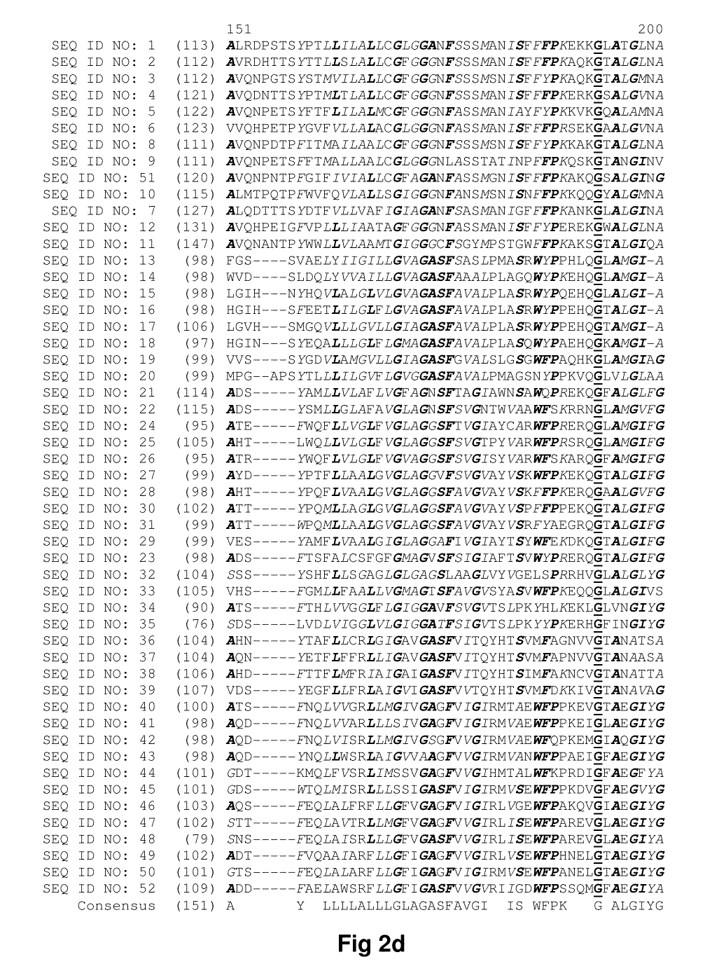

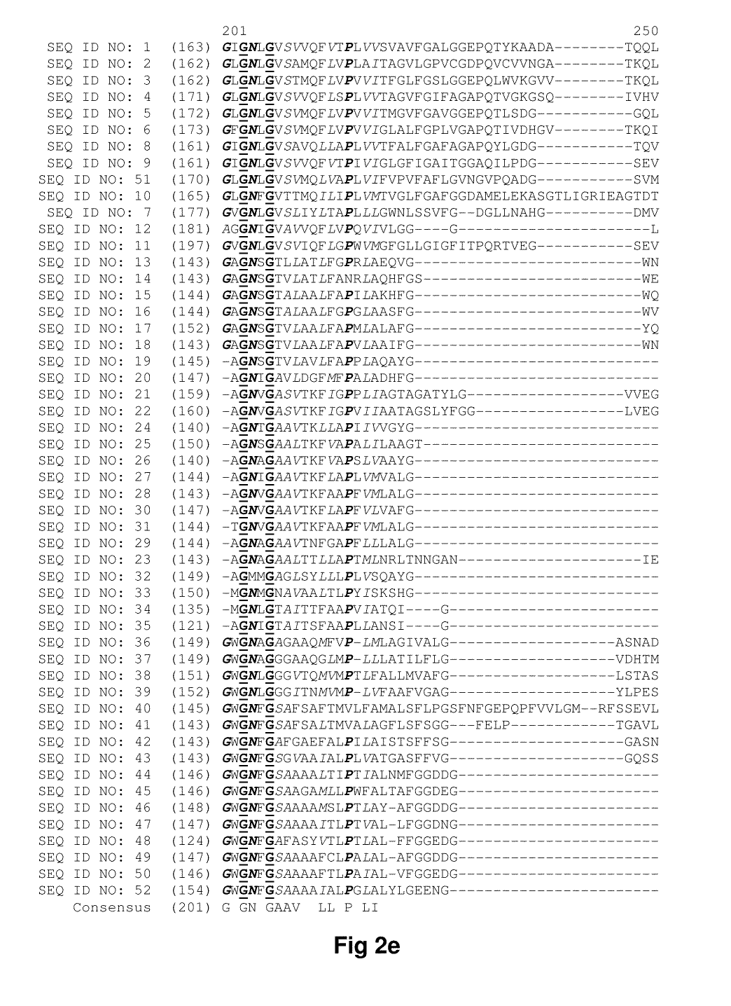

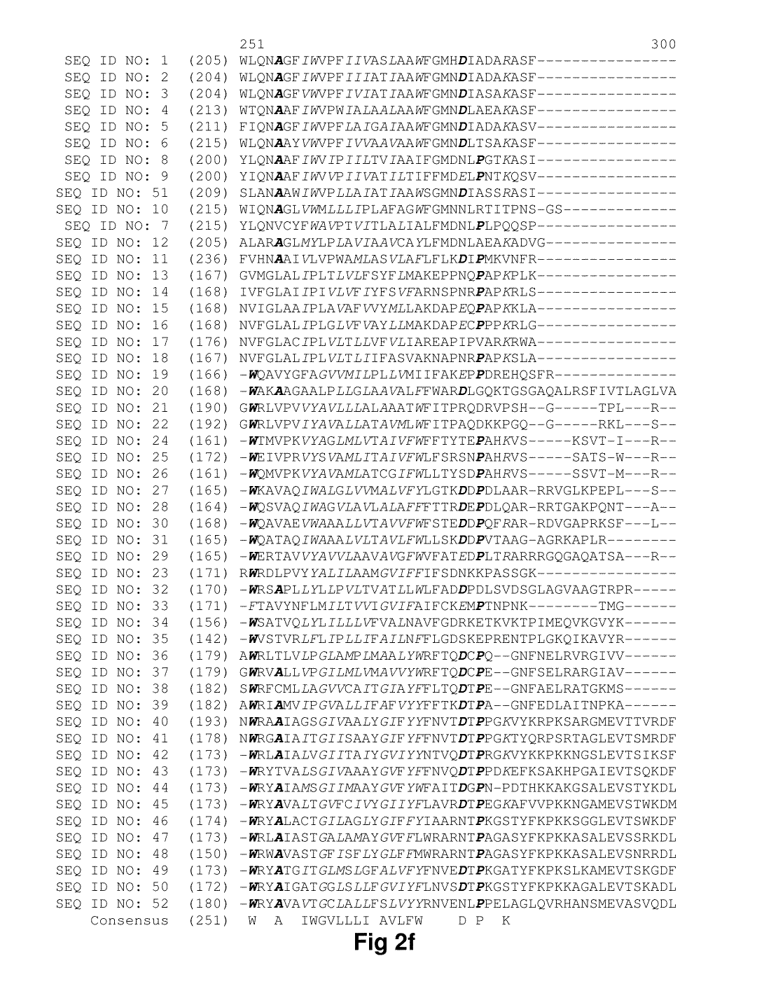

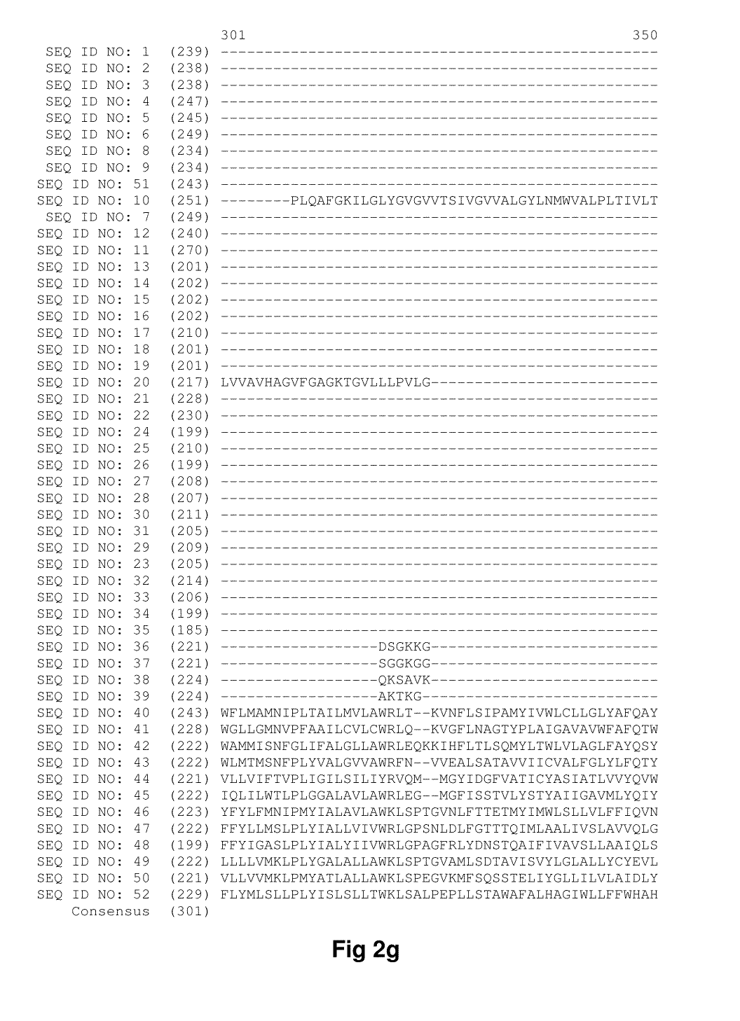

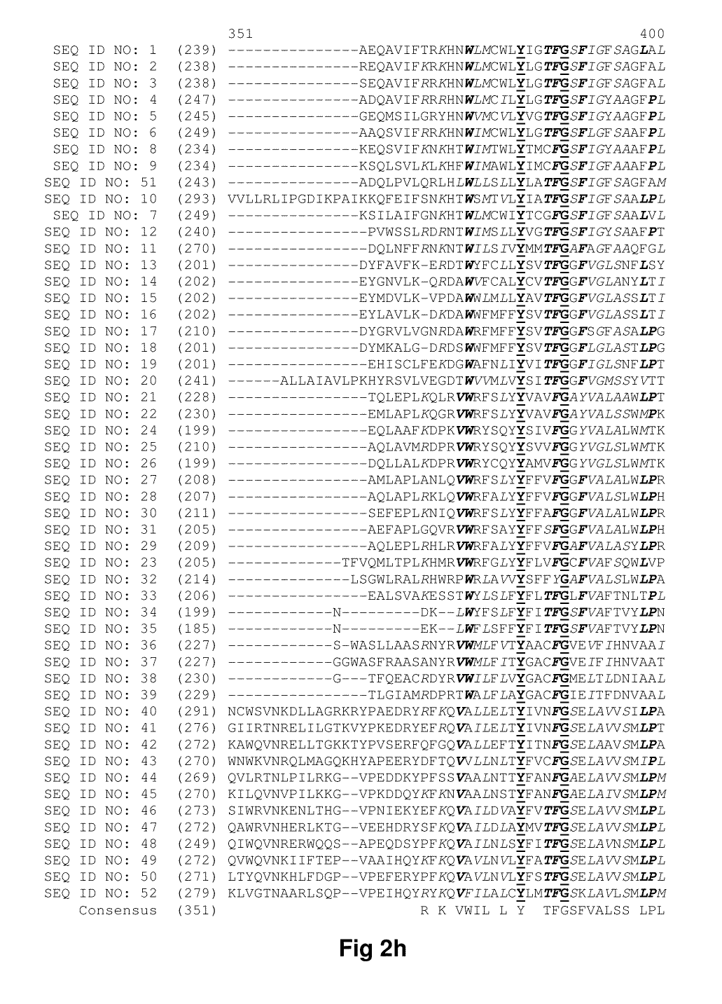

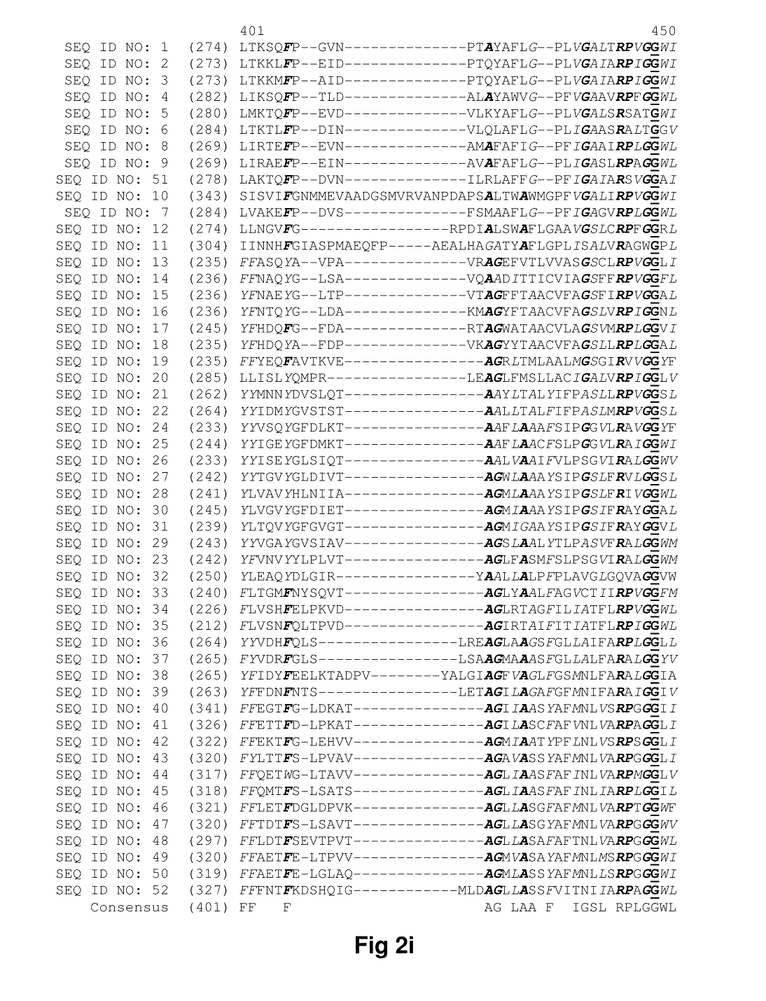

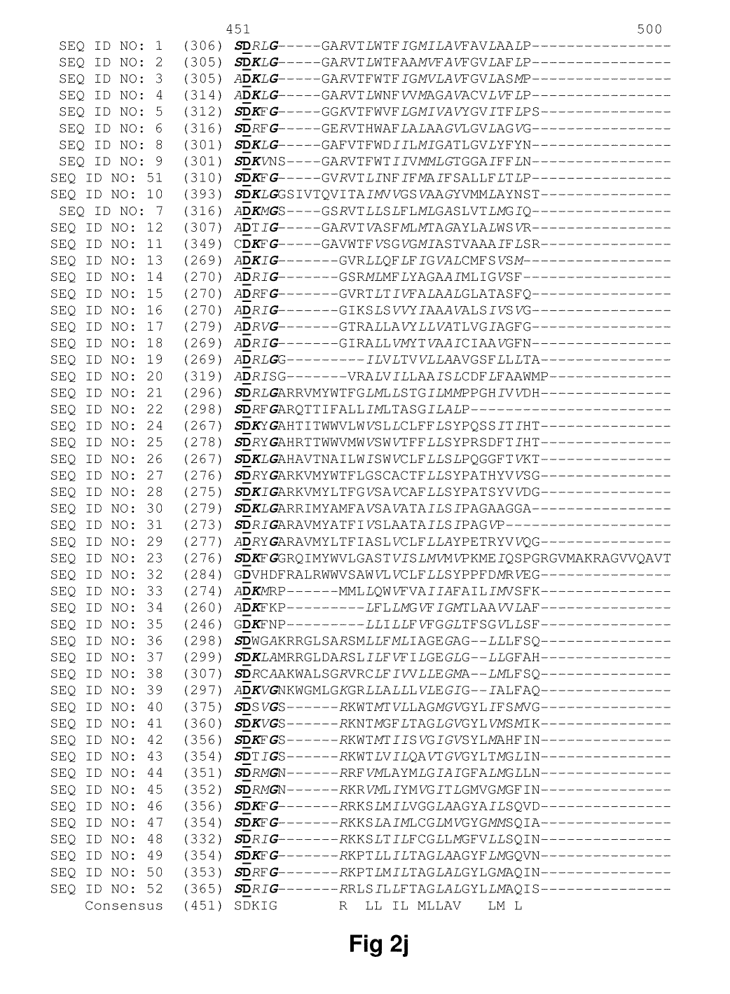

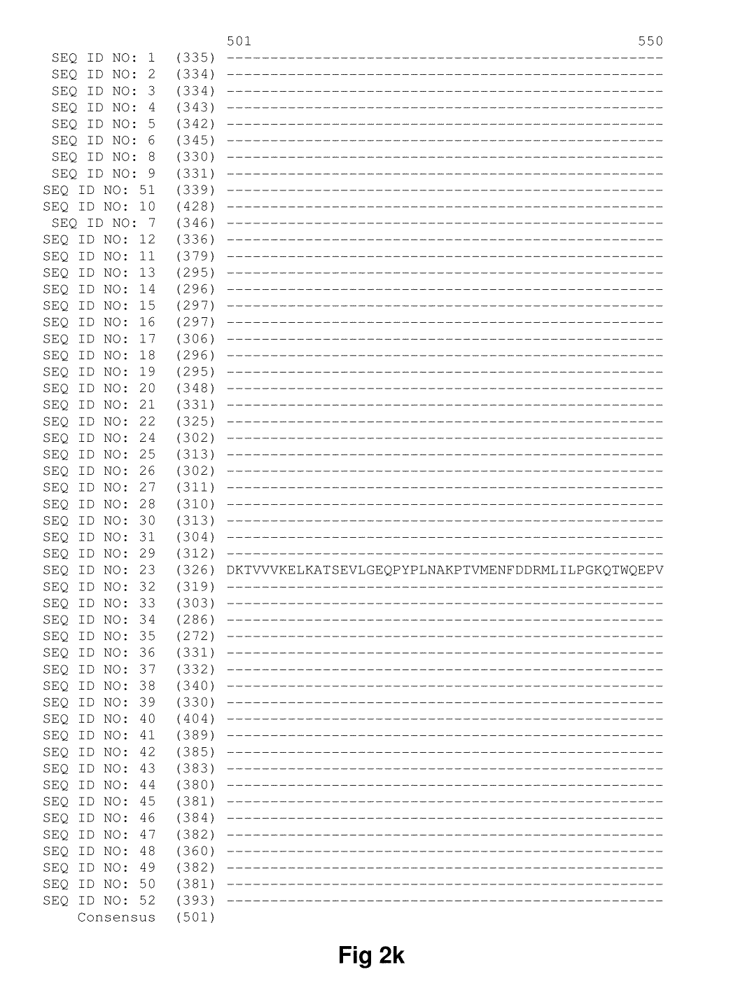

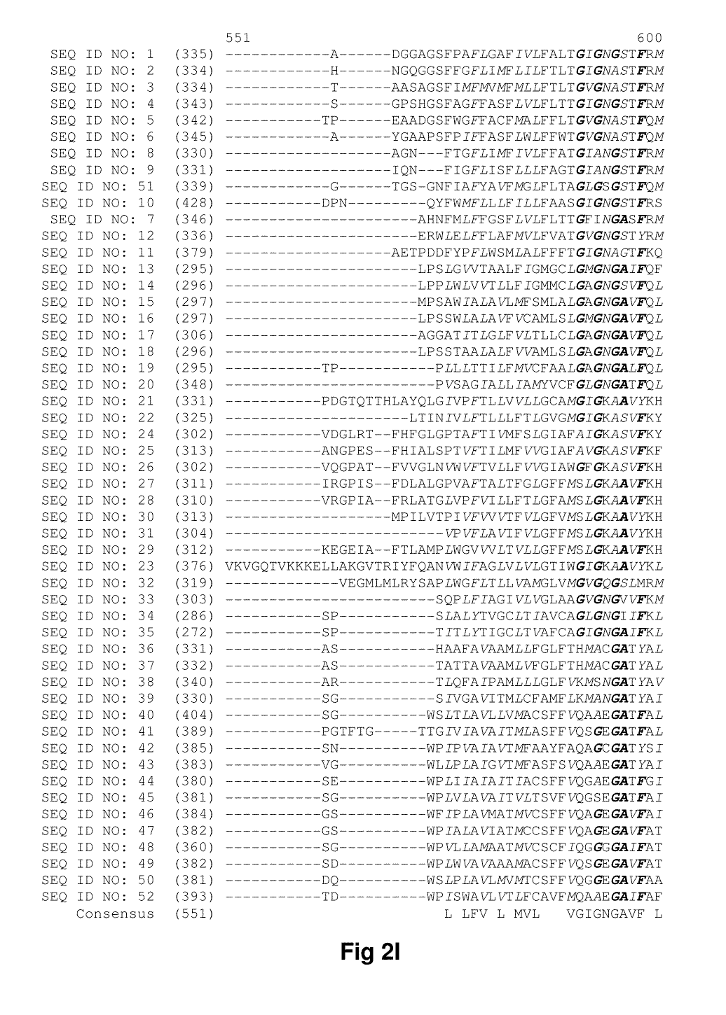

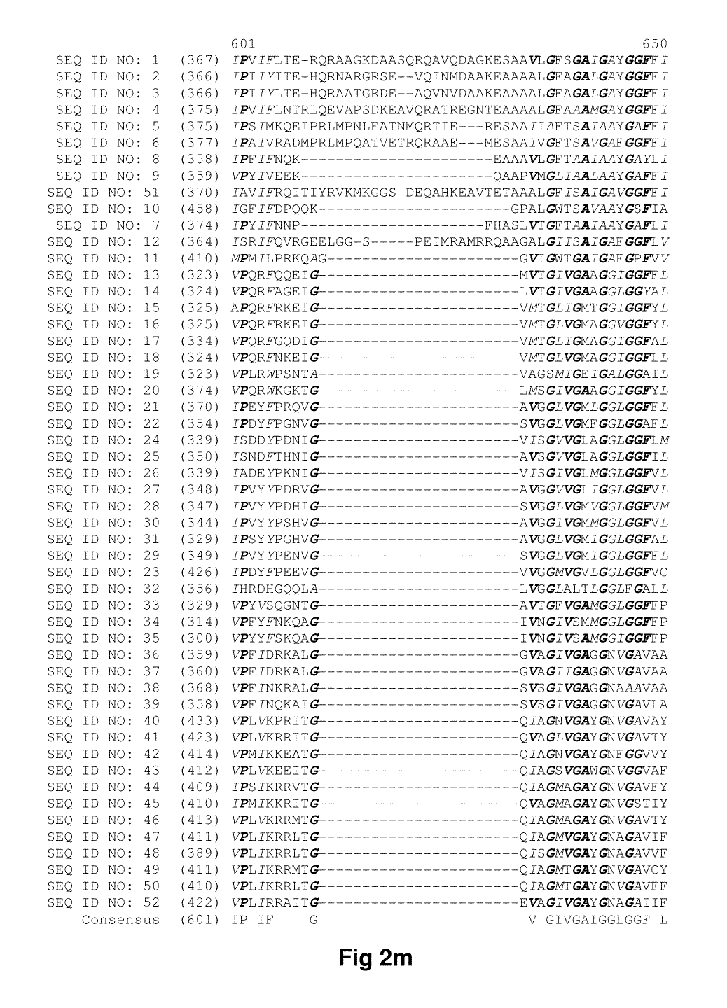

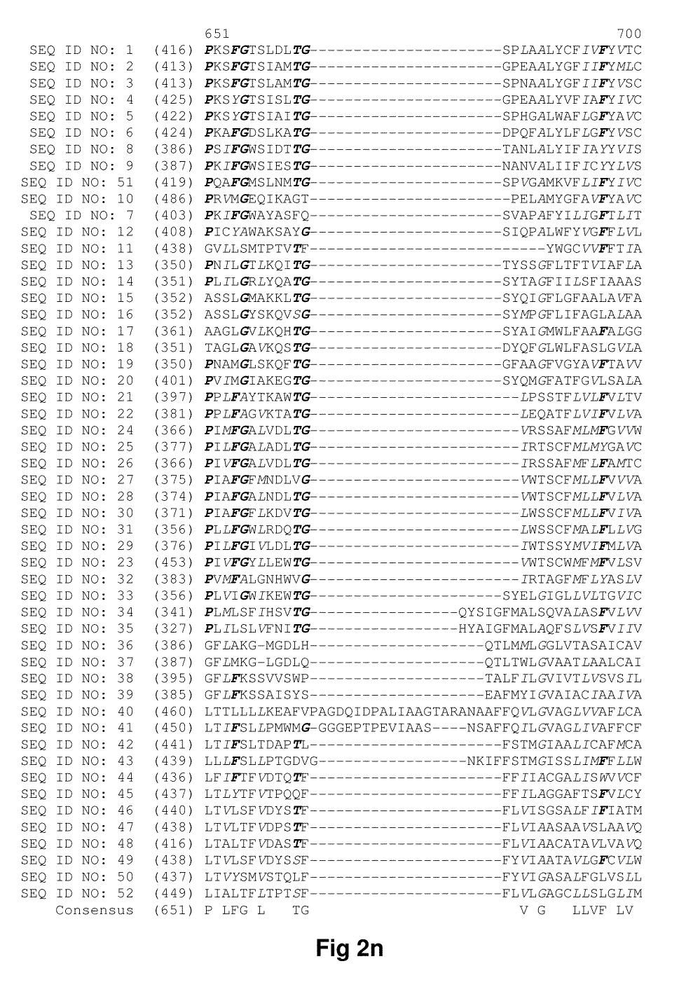

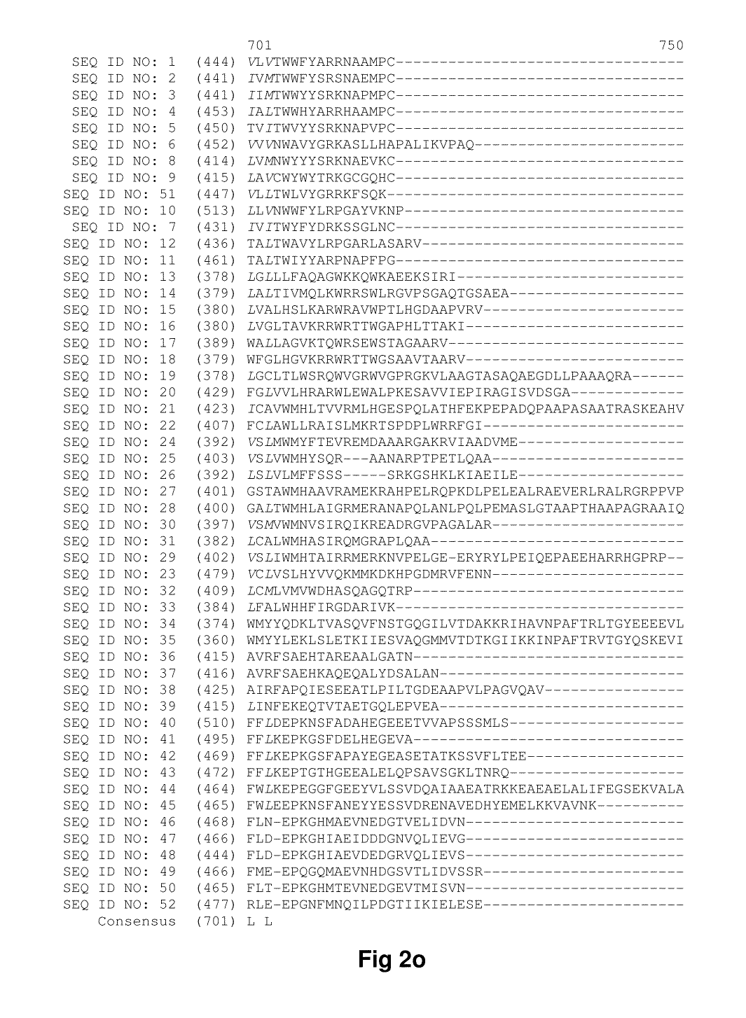

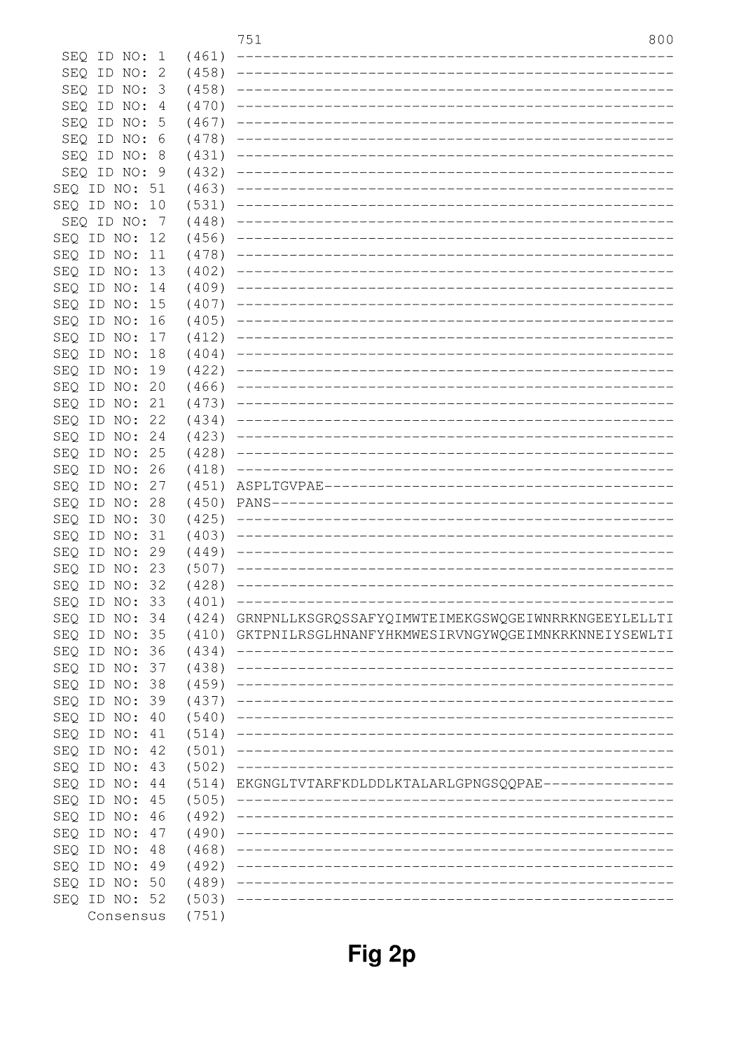

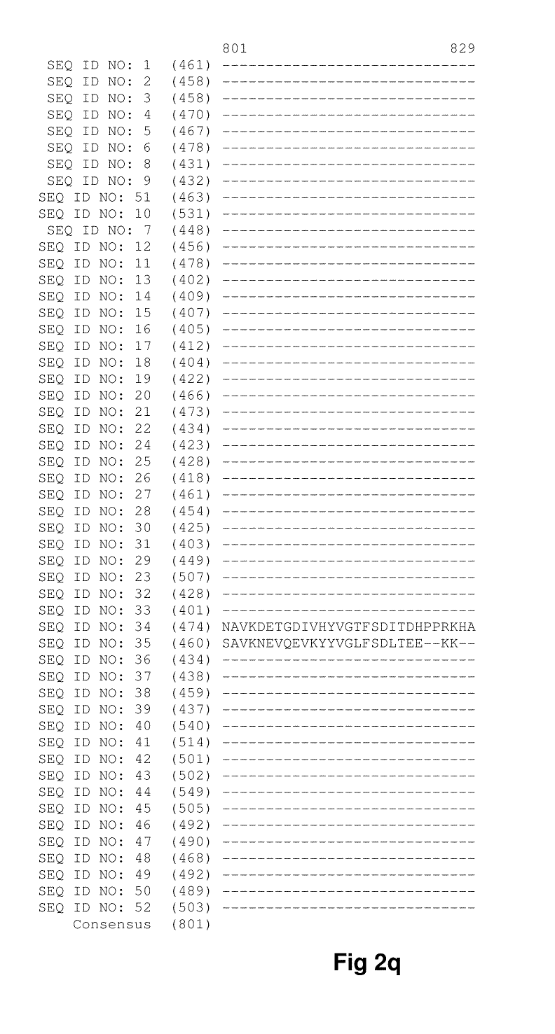

The current disclosed bacterial polypeptides from a variety of bacterial strains have common characteristics with MFS. The 11-residue containing MFS-specific sequence motif, GXXX(D/E)(R/K)XGX(R/K)(R/K) between transmembrane domain 2 and 3, was identified by Jessen-Marshall et al. using the lactose permease of E. coli as a representative model system (Jessen-Marshall et al., (1995) J. Biol. Chem. 270:16251-16257). The 52 disclosed bacterial polypeptide sequence alignment is shown in FIG. 2. A conserved motif, GMLXDRFGGRX, showed in the consensus sequence with 10 out of 11 residues matching the ones in predicted MFS-specific motif.

### Example 3

### Designing Constructs

The open reading frame (ORF) of individual bacterial MFS gene was codon optimized for maize expression and synthesized. A root-specific promoter, e.g., ZmRM2 promoter or constitutive promoter e.g., ZmUBI promoter, was used to make the expression cassette with SbGKAF as a terminator. The expression cassette was flanked by Gateway cloning sites and the co-integrate vector for Agrobacterial transformation was made using Gateway technology. The sequence was modified to be regulatory friendly if an ORF containing potential toxic and/or allergen sites.

### Example 4

### Nitrate Uptake Assay of Bacterial MFS Genes in Yeast

To test if bacterial MFS genes are functional in eukaryotic organisms, well-characterized bacterial nitrate transporters, e.g., NarK from E. coli (GenBank #NC_{—}000913) and NasA from Bacillus subtilis (Genbank #AL009126), were codon optimized for Pichia pastoris expression, synthesized, then cloned into P. pastoris expression vectors. (U.S. Patent publication 2008/0311612). The nitrate transporter activity from NarK and NasA was detected in yeast which indicates that bacterial MFS genes are able to uptake nitrate in eukaryotes.

Fifteen disclosed bacterial MFS genes including SEQ ID NO: 4, 8, 11, 12, 19, 20, 22, 24, 32, 33, 35, 38, 42, 50 and 52 with codon optimized for maize expression were evaluated in P. pastoris system for nitrate transporter activity. However, the nitrate uptake activity was undetectable which could be due to the difference of codon usage preference between maize and Pichia. The nitrate transporter assay on selected bacterial MFS genes with codon optimized for P. pastoris expression will reveal more complete information.

### Example 5

### T1 Reproductive Assay of Gaspe Flint Derived Maize Lines Under Water Limiting Conditions

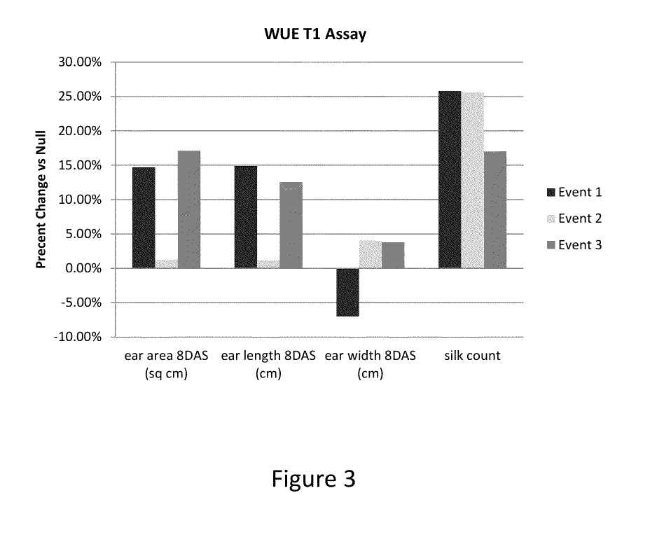

Three events carrying PHP50688 (ZmRM2:ADHI Intron:NRT2.2 (BP) (SEQ ID NO: 4)) with GS3/GF3/GF3 background were also selected for T1 water use efficiency (WUE) reproductive assay under limited water application (75% reduced water). A split block design with stationary blocks was used to minimize spatial variation. For each event, the planting of 15 transgene positive seeds and 15 respective negative seeds were completely randomized within each event block. The seeds were planted in 4-inch pots containing 50% Turface and 50% SB300 soil mixture. Drought stress was applied by delivering a minimal amount of liquid fertilizer daily for an extended period of time. Ear shoot development was monitored and the ear shoots were covered with a shoot bag to prevent pollination at the first day of silk-exertion. The un-pollinated immature ears were hand harvested at 8 days after initial silking and analyzed by digital image. Various image processing operations may be performed, e.g., techniques or algorithms to delineate image pixels associated with the immature ear object of interest from the general image background and/or extraneous debris. Data information can be recorded for each whole or subsection of immature ear objects including, without limitation, object area, minor axis length, major axis length, perimeter, ear color and/or other information regarding ear size, shape, morphology, location or color. Results are analyzed for statistical significance by comparing transgenic positives vs the respective nulls. Significant increase in immature ear parameters or vegetative parameters indicates increased draught tolerance. Some transgenic positive plants expressing NRT2.2 (BP) (SEQ ID NO: 4) tend to have significant increased ear length, and/or silk numbers compared to non-transgenic nulls (FIG. 3).

### Example 6

### T1 Reproductive Assay of Gaspe Flint Derived Maize Lines Under Nitrogen Limiting Conditions

PHP50688 (ZmRM2:ADHI Intron:NRT2.2 (BP) (SEQ ID NO: 4), PHP50692 (ZmRM2:ADHI Intron:NRT2.1 (MN) (SEQ ID NO: 19), PHP50693 (ZmRM2:ADHI Intron:NRT2.1 (BP) (SEQ ID NO: 20), and PHP50697 (ZmRM2:ADHI Intron:NRT2.1 (PL) (SEQ ID NO: 38) were selected for T1 nitrogen use efficiency (NUE) reproductive assay under limited nitrate application (4 mm nitrate).

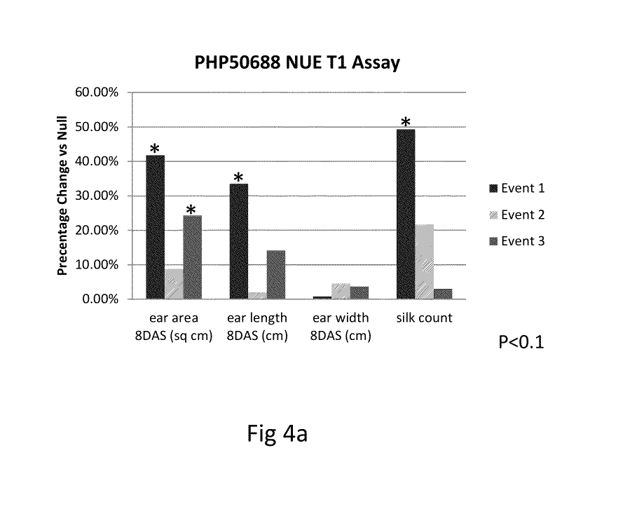

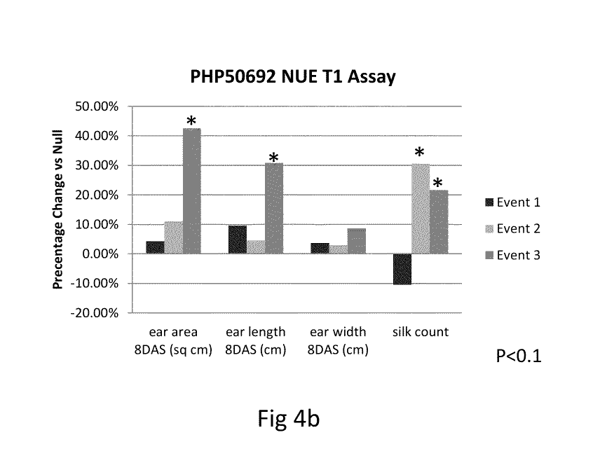

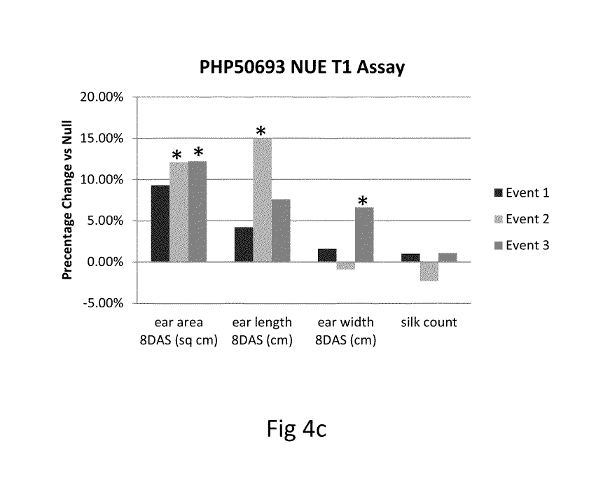

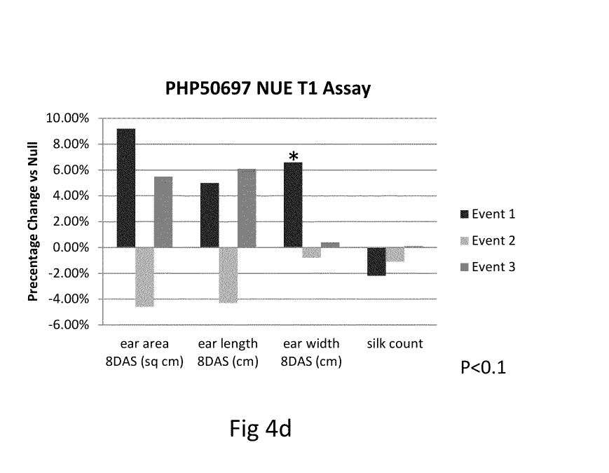

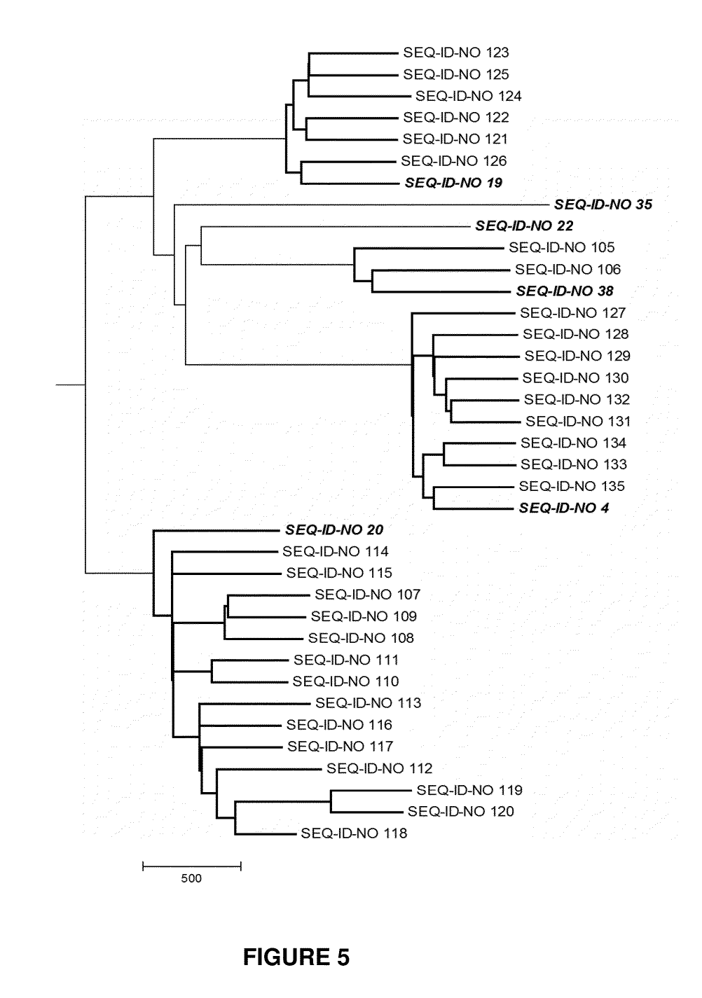

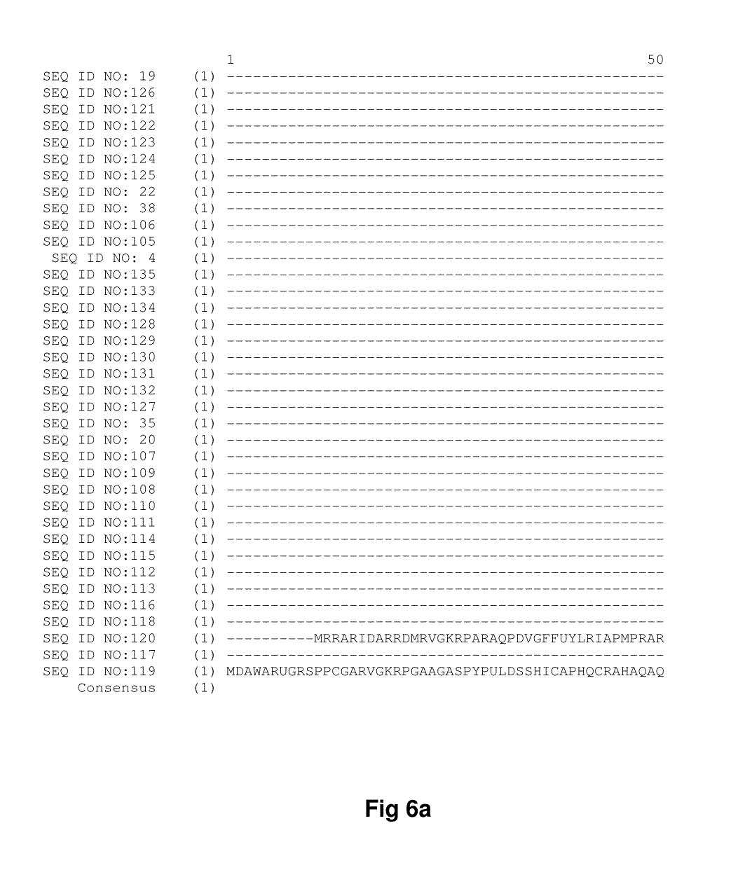

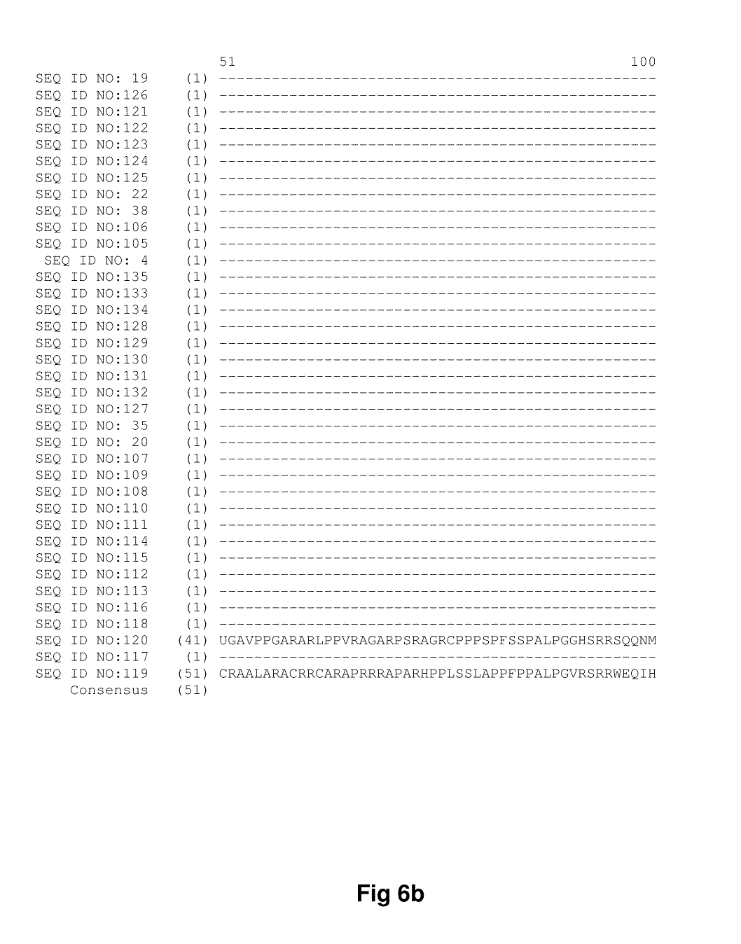

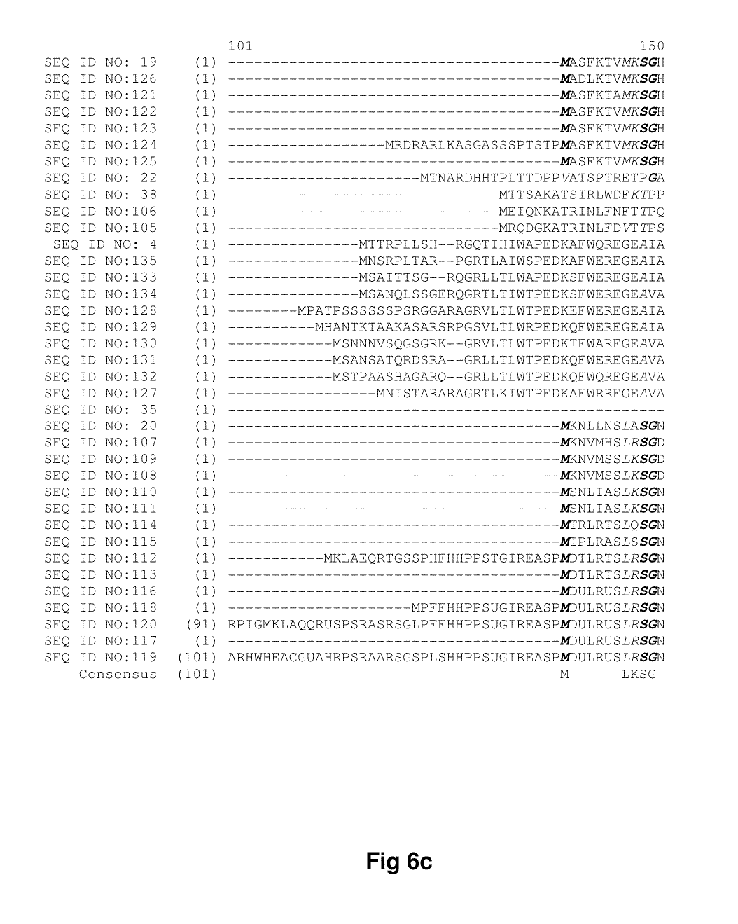

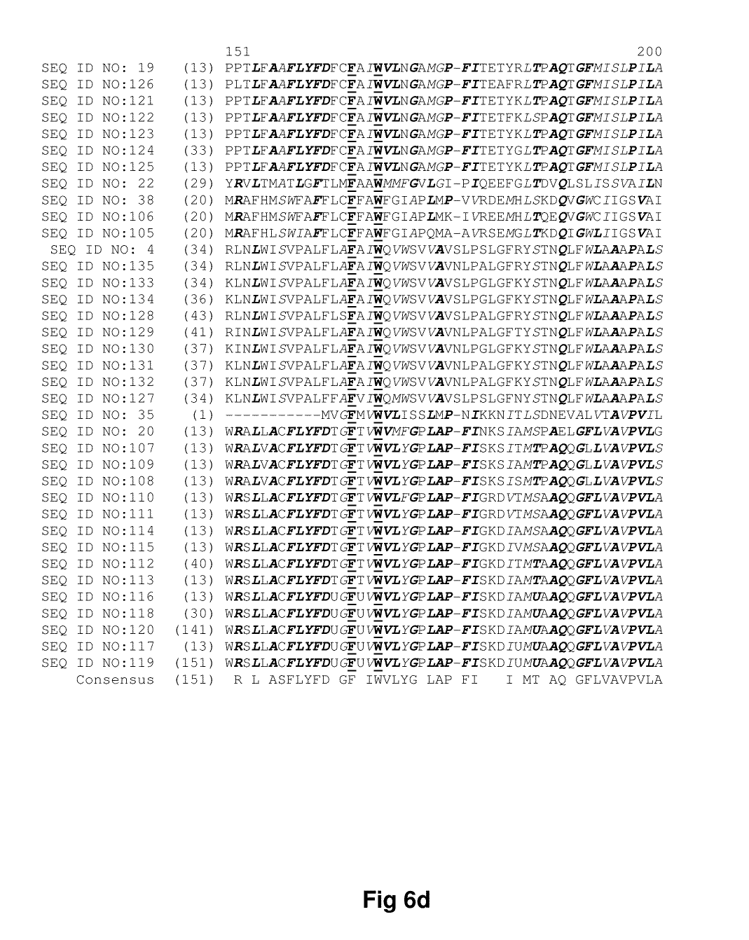

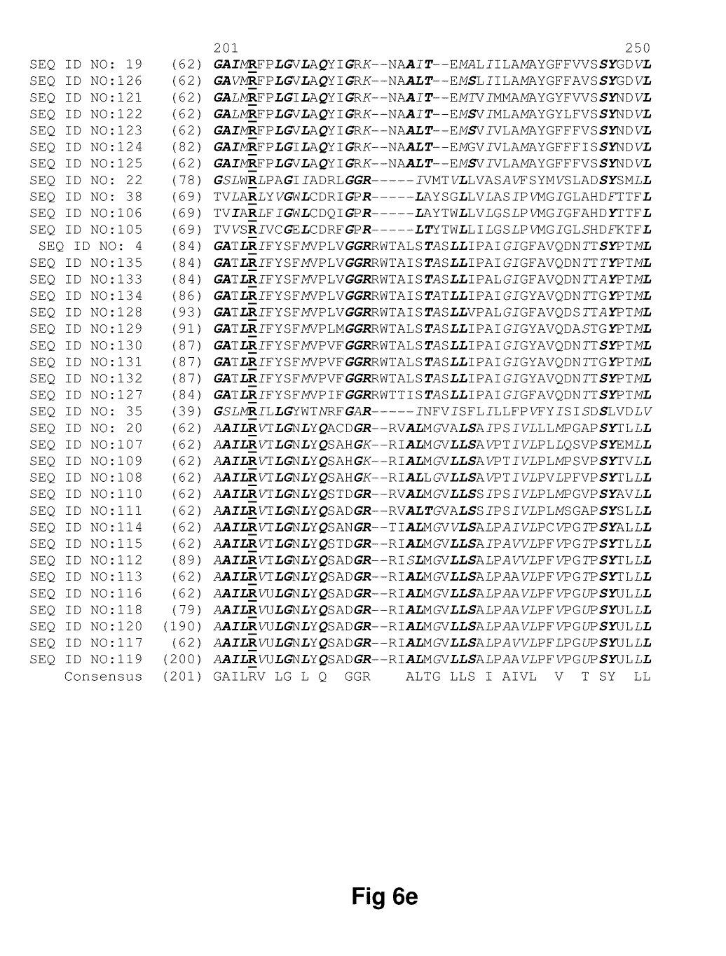

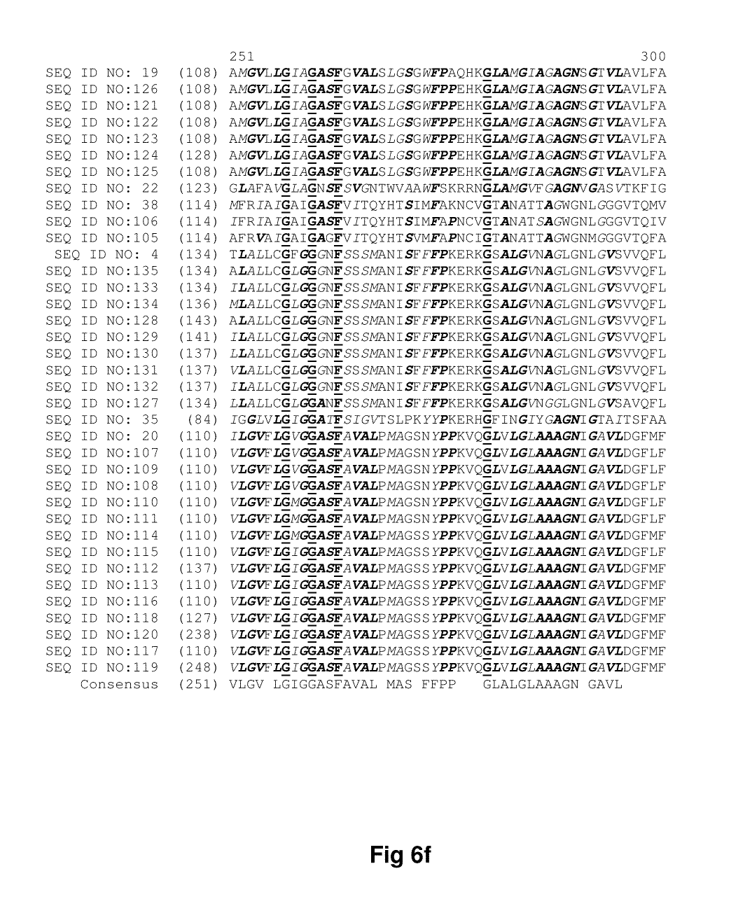

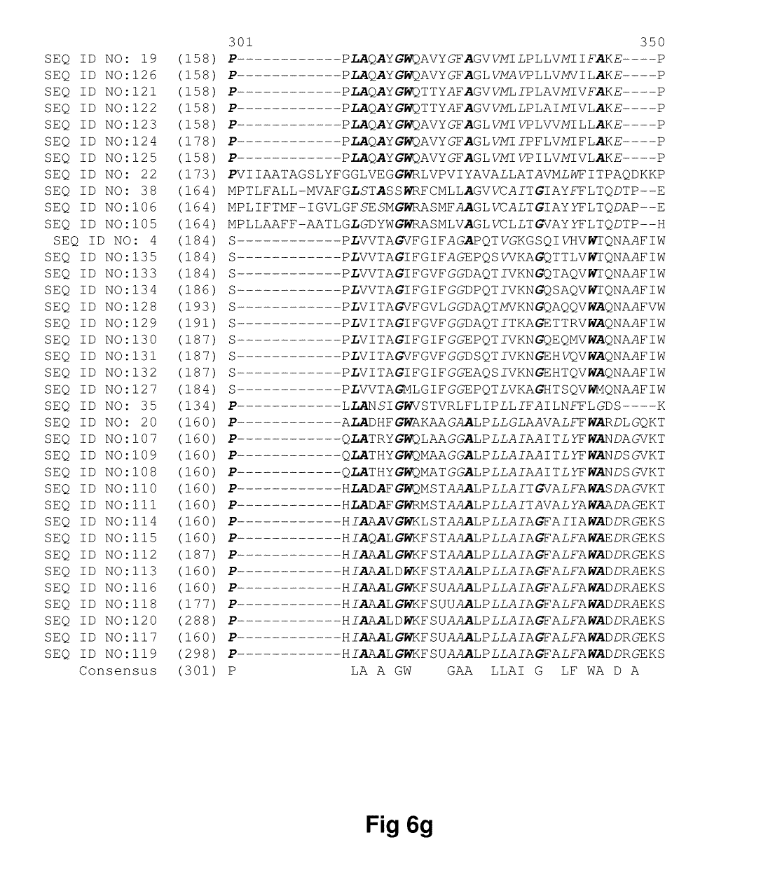

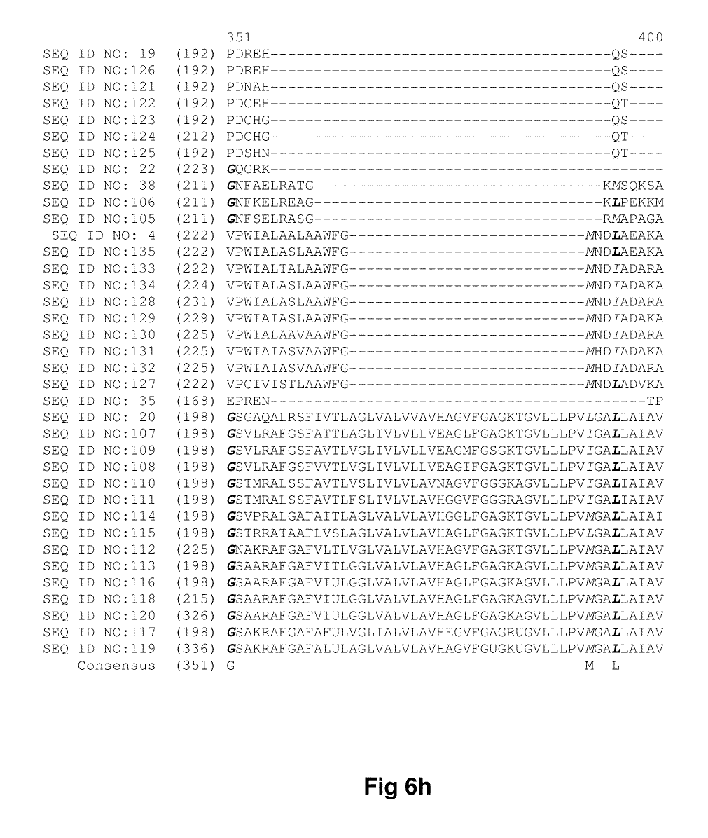

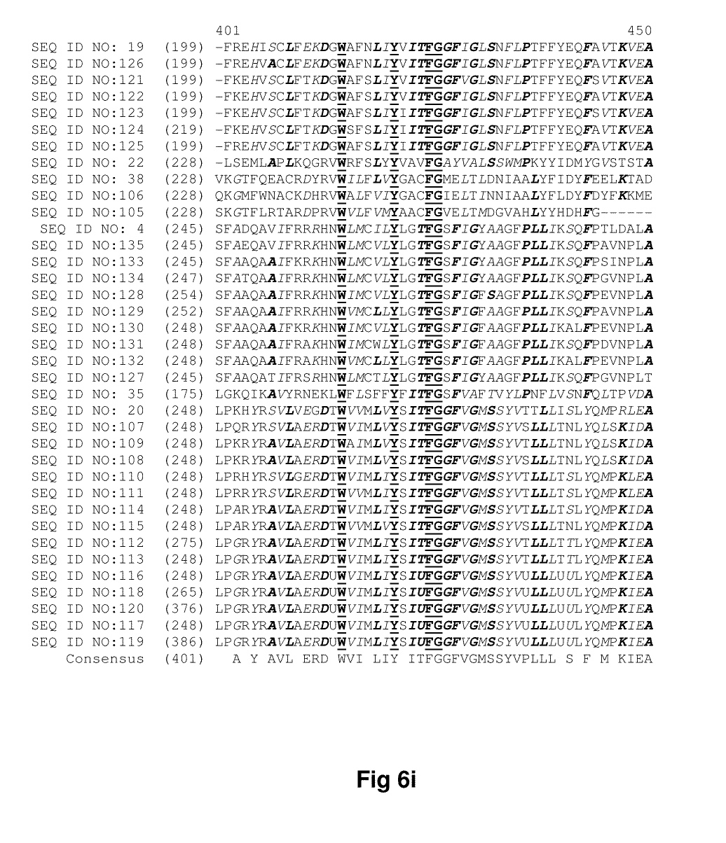

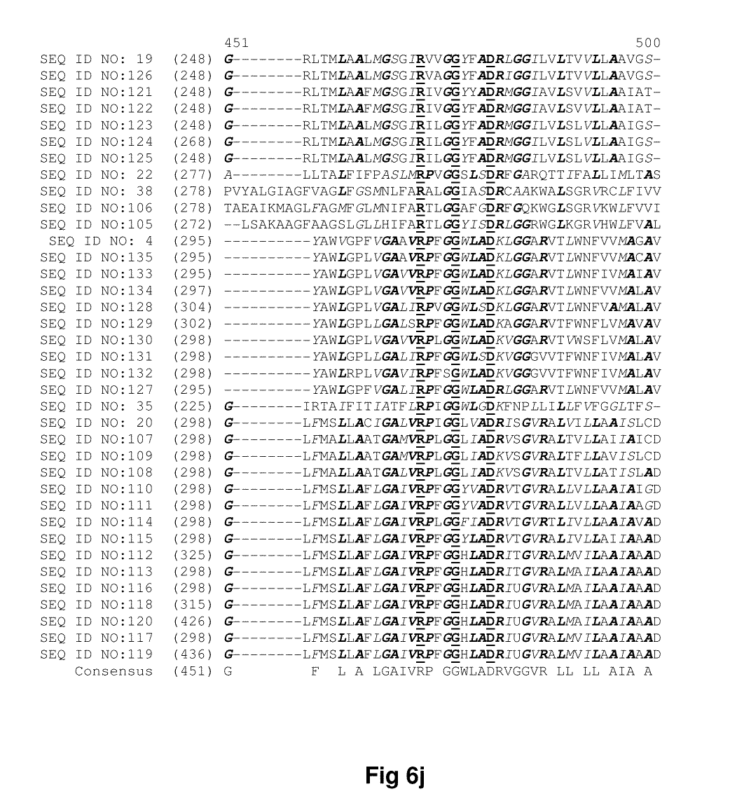

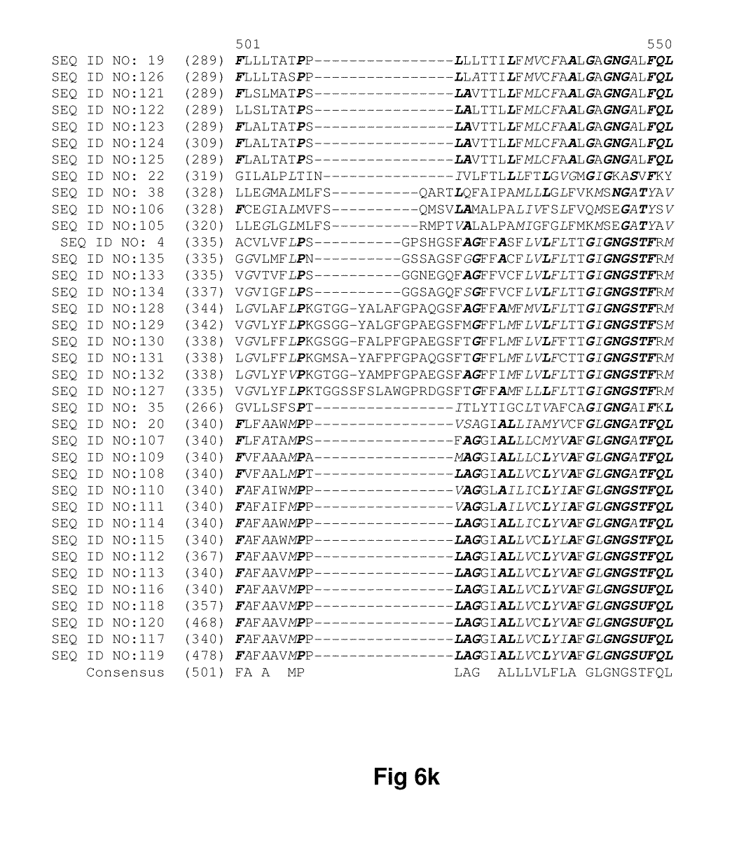

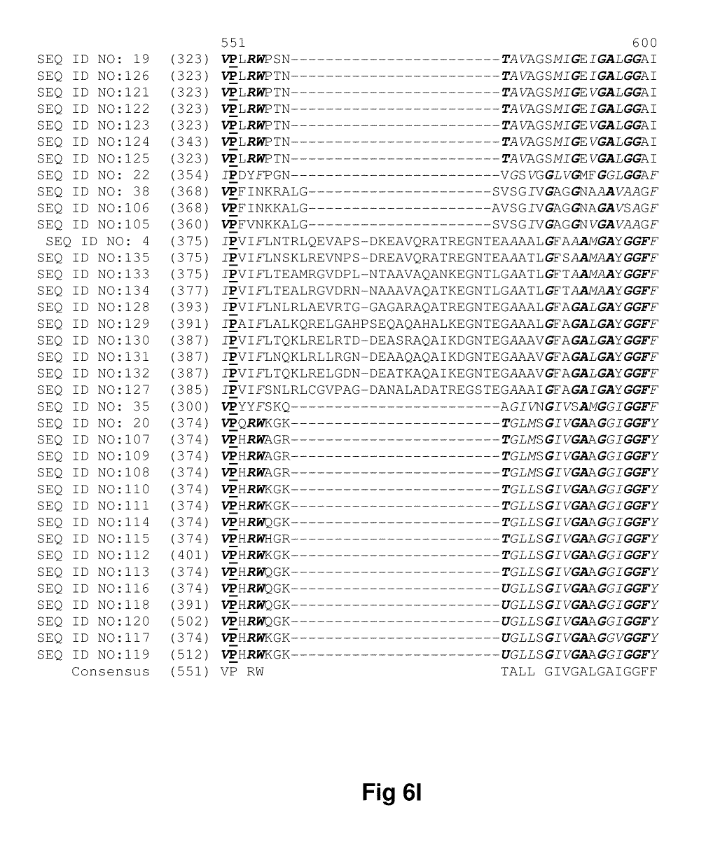

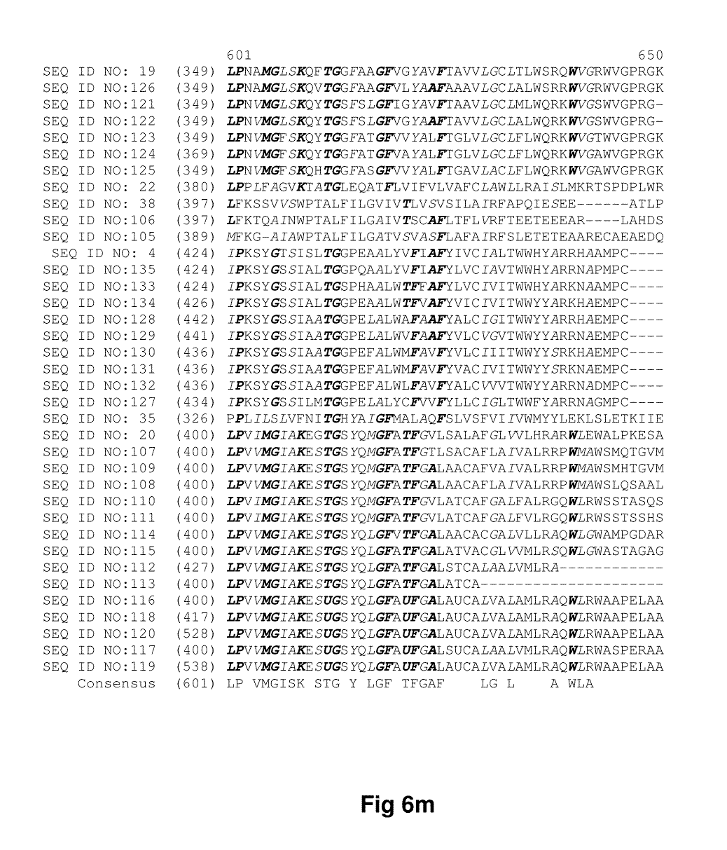

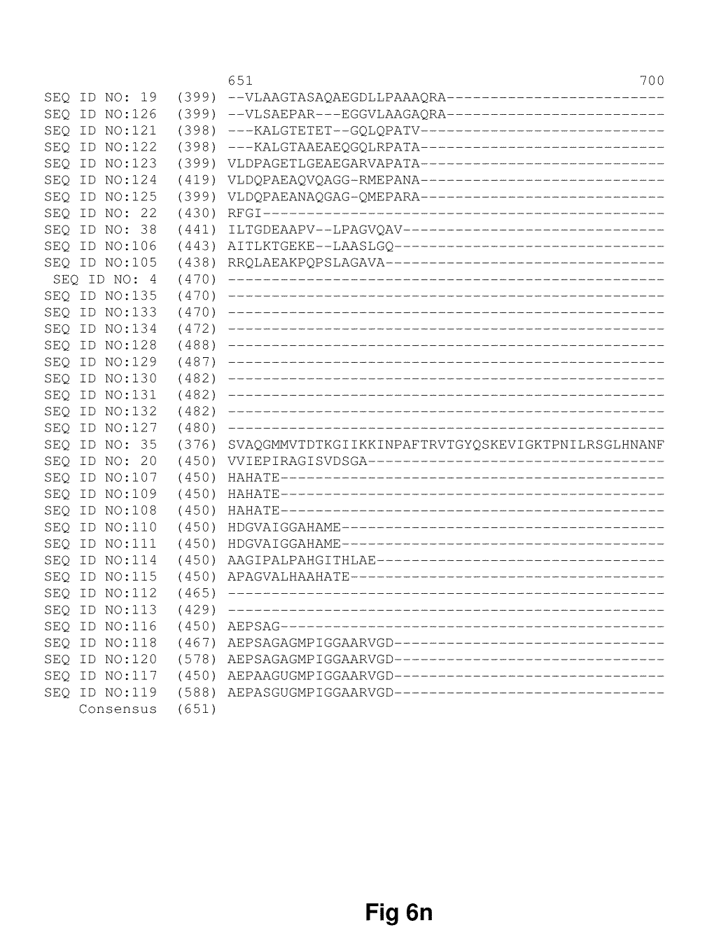

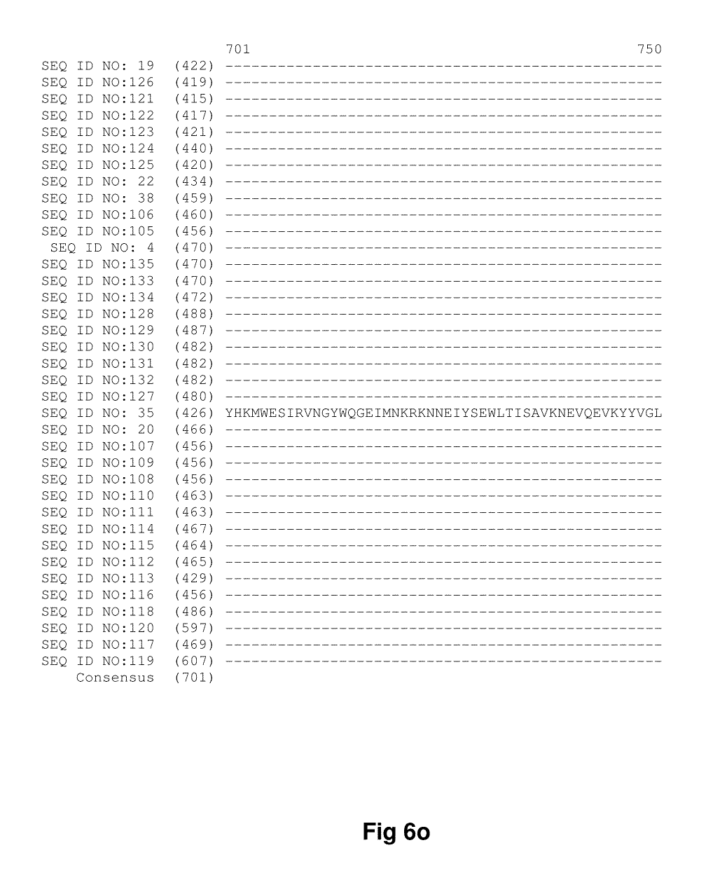

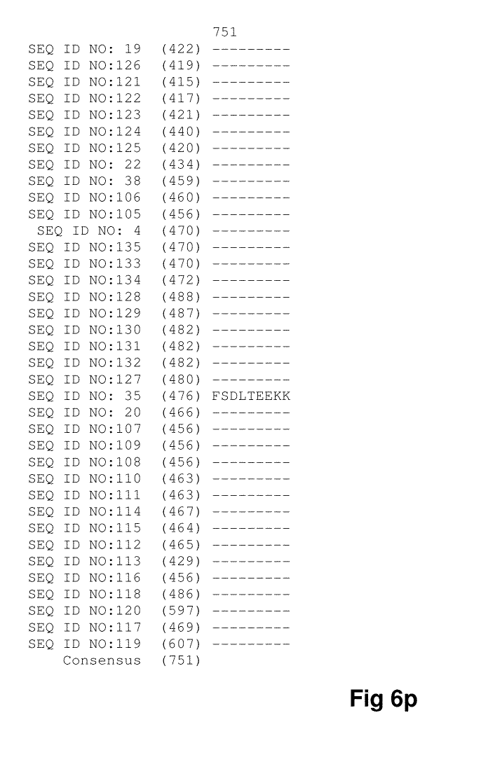

Three events from individual construct with GS3/GF3/GF3 background were assayed. A split block design with stationary blocks was used to minimize spatial variation. For each event, the planting of 15 transgene positive seeds and 15 respective negative seeds were completely randomized within each event block. The seeds were planted in 4-inch pots containing TURFACE®, a commercial potting medium and watered four times each day with 4 mm KNO₃ growth medium. Ear shoot development was monitored and the ear shoots were covered with a shoot bag to prevent pollination at the first day of silk-exertion. The un-pollinated immature ears were hand harvested at 8 days after initial sulking and analyzed by digital image. Various image processing operations may be performed, e.g., techniques or algorithms to delineate image pixels associated with the immature ear object of interest from the general image background and\or extraneous debris. Data information can be recorded for each whole or subsection of immature ear objects including, without limitation, object area, minor axis length, major axis length, perimeter, ear color and/or other information regarding ear size, shape, morphology, location or color. Results are analyzed for statistical significance by comparing transgenic positives vs the respective nulls. Significant increase in immature ear parameters or vegetative parameters indicates increased nitrogen use efficacy. Transgenic positive plants expressing PHP50688 (ZmRM2:ADHI Intron:NRT2.2 (BP) (FIG. 4A), PHP50692 (ZmRM2:ADHI Intron:NRT2.1 (MN) (FIG. 4B), PHP50693 (ZmRM2:ADHI Intron:NRT2.1 (BP) (FIG. 4C), and PHP50697 (ZmRM2:ADHI Intron:NRT2.1 (PL) (FIG. 4D) tend to have significant increased ear area, ear length, ear width, and/or silk numbers compared to non-transgenic nulls (FIG. 4).

### Example 7

### Nitrogen Use Efficiency (NUE) Assay

Seeds of Arabidopsis thaliana (control and transgenic line), ecotype Columbia, are surface sterilized (Sanchez, et al., 2002) and then plated on to Murashige and Skoog (MS) medium containing 0.8% (w/v) Bacto™-Agar (Difco). Plates are incubated for 3 days in darkness at 4° C. to break dormancy (stratification) and transferred thereafter to growth chambers (Conviron, Manitoba, Canada) at a temperature of 20° C. under a 16-h light/8-h dark cycle. The average light intensity is 120 ρE/m2/s. Seedlings are grown for 12 days and then transferred to soil based pots. Potted plants are grown on a nutrient-free soil LB2 Metro-Mix® 200 (Scott's Sierra Horticultural Products, Marysville, Ohio, USA) in individual 1.5-in pots (Arabidopsis system; Lehle Seeds, Round Rock, Tex., USA) in growth chambers, as described above. Plants are watered with 0.6 or 6.5 mM potassium nitrate in the nutrient solution based on Murashige and Skoog (MS free Nitrogen) medium. The relative humidity is maintained around 70%. Sixteen to eighteen days later, plant shoots are collected for evaluation of biomass and SPAD (chlorophyll) readings.

### Example 8

### Sucrose Growth Assay

The Columbia line of Arabidopsis thaliana is obtained from the Arabidopsis Biological Resource Center (Columbus, Ohio). For early analysis (Columbia and T3 transgenic lines), seed are surface-sterilized with 70% ethanol for 5 minutes followed by 40% Clorox® for 5 minutes and rinsed with sterile deionized water. Surface-sterilized seed are sown onto square Petri plates (25 cm) containing 95 mL of sterile medium consisting of 0.5 Murashige and Skoog (1962) salts (Life Technologies) and 4% (w/v) phytagel (Sigma). The medium contains no supplemental sucrose. Sucrose is added to medium in 0.1%, 0.5% and 1.5% concentration. Plates are arranged vertically in plastic racks and placed in a cold room for 3 days at 4° C. to synchronize germination. Racks with cold stratified seed are then transferred into growth chambers (Conviron, Manitoba, Canada) with day and night temperatures of 22 and 20° C., respectively. The average light intensity at the level of the rosette is maintained at 110 mol/m2/secl during a 16-hr light cycle development beginning at removal from the cold room (day 3 after sowing) until the seedlings are harvested on day 14. Images are taken and total fresh weight of root and shoot are measured.

### Example 9

### Low Nitrogen Seedling Assay Protocol

Seed of transgenic events are separated into transgene and null seed. Two different random assignments of treatments are made to each block of 54 pots arranged 6 rows of 9 columns using 9 replicates of all treatments. In one case null seed of 5 events of the same construct are mixed and used as control for comparison of the 5 positive events in this block, making up 6 treatment combinations in each block. In the second case, 3 transgenic positive treatments and their corresponding nulls are randomly assigned to the 54 pots of the block, making 6 treatment combinations for each block, containing 9 replicates of all treatment combinations. In the first case transgenic parameters are compared to a bulked construct null and in the second case transgenic parameters are compared to the corresponding event null. In cases where there are 10, 15 or 20 events in a construct, the events are assigned in groups of 5 events, the variances calculated for each block of 54 pots but the block null means pooled across blocks before mean comparisons are made.

Two seed of each treatment are planted in 4 inch, square pots containing TURFACE®-MVP on 8 inch, staggered centers and watered four times each day with a solution containing the following nutrients:

| | | | |
|---|---|---|---|
| 1 mM CaCl₂ | 2 mM MgSO₄ | 0.5 mM KH₂PO₄ | 83 ppm Sprint330 |
| 3 mM KCl | 1 mM KNO₃ | 1 uM ZnSO₄ | 1 uM MnCl₂ |
| 3 uM H₃BO₄ | 1 uM MnCl₂ | 0.1 uM CuSO₄ | 0.1 uM NaMoO₄ |

After emergence the plants are thinned to one seed per pot. Seedlings are harvested 18 days after planting. At harvest, plants are removed from the pots and the Turface washed from the roots. The roots are separated from the shoot, placed in a paper bag and dried at 70° C. for 70 hr. The dried plant parts (roots and shoots) are weighed and placed in a 50 ml conical tube with approximately 20 5/32 inch steel balls and ground by shaking in a paint shaker. Approximately, 30 mg of the ground tissue is hydrolyzed in 2 ml of 20% H₂O₂ and 6M H₂SO₄ for 30 minutes at 170° C. After cooling, water is added to 20 ml, mixed thoroughly, and a 50 μl aliquot removed and added to 950 μl 1M Na₂CO₃. The ammonia in this solution is used to estimate total reduced plant nitrogen by placing 100 μl of this solution in individual wells of a 96 well plate followed by adding 50 μl of OPA solution. Fluorescence, excitation=360 nM/emission=530 nM, is determined and compared to NH₄Cl standards dissolved in a similar solution and treated with OPA solution.

OPA solution—5 ul Mercaptoethanol+1 ml OPA stock solution
OPA stock—50 mg o-phthadialdehyde (OPA—Sigma #P0657) dissolved in 1.5 ml methanol+4.4 ml 1M Borate buffer pH9.5 (3.09 g H₃BO₄+1 g NaOH in 50 ml water)+0.55 ml 20% SDS

The following parameters are measured and means compared to null mean parameters using a Student's t test: total plant biomass; root biomass; shoot biomass; root/shoot ratio; plant N concentration; total plant N.

Variance is calculated within each block using a nearest neighbor calculation as well as by Analysis of Variance (ANOVA) using a completely random design (CRD) model. An overall treatment effect for each block is calculated using an F statistic by dividing overall block treatment mean square by the overall block error mean square.

### Example 10

### Transformation of Maize

### Biolistics

Polynucleotides contained within a vector can be transformed into embryogenic maize callus by particle bombardment, generally as described by Tomes, et al., Plant Cell, Tissue and Organ Culture: Fundamental Methods, Eds. Gamborg and Phillips, Chapter 8, pgs. 197-213 (1995) and as briefly outlined below. Transgenic maize plants can be produced by bombardment of embryogenically responsive immature embryos with tungsten particles associated with DNA plasmids. The plasmids typically comprise a selectable marker and a structural gene, or a selectable marker and a polynucleotide sequence or subsequence, or the like.

### Preparation of Particles

Fifteen mg of tungsten particles (General Electric), 0.5 to 1.8p, preferably 1 to 1.8p, and most preferably 1μ, are added to 2 ml of concentrated nitric acid. This suspension is sonicated at 0° C. for 20 minutes (Branson Sonifier Model 450, 40% output, constant duty cycle). Tungsten particles are pelleted by centrifugation at 10000 rpm (Biofuge) for one minute and the supernatant is removed. Two milliliters of sterile distilled water are added to the pellet, and brief sonication is used to resuspend the particles. The suspension is pelleted, one milliliter of absolute ethanol is added to the pellet and brief sonication is used to resuspend the particles. Rinsing, pelleting and resuspending of the particles are performed two more times with sterile distilled water and finally the particles are resuspended in two milliliters of sterile distilled water. The particles are subdivided into 250-μl aliquots and stored frozen.

### Preparation of Particle-Plasmid DNA Association

The stock of tungsten particles are sonicated briefly in a water bath sonicator (Branson Sonifier Model 450, 20% output, constant duty cycle) and 50 μl is transferred to a microfuge tube. The vectors are typically cis: that is, the selectable marker and the gene (or other polynucleotide sequence) of interest are on the same plasmid.

Plasmid DNA is added to the particles for a final DNA amount of 0.1 to 10 μg in 10 μL total volume and briefly sonicated. Preferably, 10 μg (1 μg/μL in TE buffer) total DNA is used to mix DNA and particles for bombardment. Fifty microliters (50 μL) of sterile aqueous 2.5 M CaCl₂ are added and the mixture is briefly sonicated and vortexed. Twenty microliters (20 μL) of sterile aqueous 0.1 M spermidine are added and the mixture is briefly sonicated and vortexed. The mixture is incubated at room temperature for 20 minutes with intermittent brief sonication. The particle suspension is centrifuged and the supernatant is removed. Two hundred fifty microliters (250 μL) of absolute ethanol are added to the pellet, followed by brief sonication. The suspension is pelleted, the supernatant is removed and 60 μl of absolute ethanol are added. The suspension is sonicated briefly before loading the particle-DNA agglomeration onto macrocarriers.

### Preparation of Tissue

Immature embryos of maize variety High Type II are the target for particle bombardment-mediated transformation. This genotype is the F1 of two purebred genetic lines, parents A and B, derived from the cross of two known maize inbreds, A188 and B73. Both parents were selected for high competence of somatic embryogenesis, according to Armstrong, et al., (1991) Maize Genetics Coop. News 65:92.

Ears from F1 plants are selfed or sibbed and embryos are aseptically dissected from developing caryopses when the scutellum first becomes opaque. This stage occurs about 9 to 13 days post-pollination and most generally about 10 days post-pollination, depending on growth conditions. The embryos are about 0.75 to 1.5 millimeters long. Ears are surface sterilized with 20% to 50% Clorox® for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite® and 8.5 mg/l AgNO₃ Chu, et al., (1975) Sci. Sin. 18:659; Eriksson, (1965) Physiol. Plant 18:976. The medium is sterilized by autoclaving at 121° C. for 15 minutes and dispensed into 100×25 mm Petri dishes. AgNO₃ is filter-sterilized and added to the medium after autoclaving. The tissues are cultured in complete darkness at 28° C. After about 3 to 7 days, most usually about 4 days, the scutellum of the embryo swells to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3 to 16 hours, preferably 4 hours, in complete darkness at 28° C. just prior to bombardment with particles associated with plasmid DNA.

To effect particle bombardment of embryos, the particle-DNA agglomerates are accelerated using a DuPont PDS-1000 particle acceleration device. The particle-DNA agglomeration is briefly sonicated and 10 μl are deposited on macrocarriers and the ethanol is allowed to evaporate. The macrocarrier is accelerated onto a stainless-steel stopping screen by the rupture of a polymer diaphragm (rupture disk). Rupture is affected by pressurized helium. The velocity of particle-DNA acceleration is determined based on the rupture disk breaking pressure. Rupture disk pressures of 200 to 1800 psi are used, with 650 to 1100 psi being preferred and about 900 psi being most highly preferred. Multiple disks are used to affect a range of rupture pressures.

The shelf containing the plate with embryos is placed 5.1 cm below the bottom of the macrocarrier platform (shelf #3). To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 in Hg. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/l thiamine HCl, 30 gm/l sucrose, 1 mg/l 2,4-dichlorophenoxyacetic acid, 2 gm/l Gelrite®, 0.85 mg/l Ag NO₃ and 3 mg/l bialaphos (Herbiace, Meiji). Bialaphos is added filter-sterilized. The embryos are subcultured to fresh selection medium at 10 to 14 day intervals. After about 7 weeks, embryogenic tissue, putatively transformed for both selectable and unselected marker genes, proliferates from a fraction of the bombarded embryos. Putative transgenic tissue is rescued and that tissue derived from individual embryos is considered to be an event and is propagated independently on selection medium. Two cycles of clonal propagation are achieved by visual selection for the smallest contiguous fragments of organized embryogenic tissue.

A sample of tissue from each event is processed to recover DNA. The DNA is restricted with a restriction endonuclease and probed with primer sequences designed to amplify DNA sequences overlapping the ZmBZIP and non-ZmBZIP portion of the plasmid. Embryogenic tissue with amplifiable sequence is advanced to plant regeneration.

For regeneration of transgenic plants, embryogenic tissue is subcultured to a medium comprising MS salts and vitamins (Murashige and Skoog, (1962) Physiol. Plant 15:473), 100 mg/l myo-inositol, 60 gm/l sucrose, 3 gm/l Gelrite®, 0.5 mg/l zeatin, 1 mg/l indole-3-acetic acid, 26.4 ng/I cis-trans-abscissic acid and 3 mg/l bialaphos in 100×25 mm Petri dishes and is incubated in darkness at 28° C. until the development of well-formed, matured somatic embryos is seen. This requires about 14 days. Well-formed somatic embryos are opaque and cream-colored and are comprised of an identifiable scutellum and coleoptile. The embryos are individually subcultured to a germination medium comprising MS salts and vitamins, 100 mg/l myo-inositol, 40 gm/l sucrose and 1.5 gm/l Gelrite® in 100×25 mm Petri dishes and incubated under a 16 hour light:8 hour dark photoperiod and 40 meinsteins⁻²sec⁻¹ from cool-white fluorescent tubes. After about 7 days, the somatic embryos germinate and produce a well-defined shoot and root. The individual plants are subcultured to germination medium in 125×25 mm glass tubes to allow further plant development. The plants are maintained under a 16 hour light: 8 hour dark photoperiod and 40 meinsteinsm⁻²sec⁻¹ from cool-white fluorescent tubes. After about 7 days, the plants are well-established and are transplanted to horticultural soil, hardened off and potted into commercial greenhouse soil mixture and grown to sexual maturity in a greenhouse. An elite inbred line is used as a male to pollinate regenerated transgenic plants.

### Agrobacterium-Mediated

For Agrobacterium-mediated transformation, the method of Zhao, et al., may be employed as in PCT Patent Publication Number WO 1998/32326, the contents of which are hereby incorporated by reference. Briefly, immature embryos are isolated from maize and the embryos contacted with a suspension of Agrobacterium (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos re cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants.

### Example 11

### Expression of Transqenes in Monocots

A plasmid vector is constructed comprising a preferred promoter operably linked to an isolated polynucleotide comprising a Bacterial MFS polynucleotide sequence or subsequence. This construct can then be introduced into maize cells by the following procedure.

Immature maize embryos are dissected from developing caryopses derived from crosses of maize lines. The embryos are isolated 10 to 11 days after pollination when they are 1.0 to 1.5 mm long. The embryos are then placed with the axis-side facing down and in contact with agarose-solidified N6 medium (Chu, et al., (1975) Sci. Sin. Peking 18:659-668). The embryos are kept in the dark at 27° C. Friable embryogenic callus, consisting of undifferentiated masses of cells with somatic proembryoids and embryoids borne on suspensor structures, proliferates from the scutellum of these immature embryos. The embryogenic callus isolated from the primary explant can be cultured on N6 medium and sub-cultured on this medium every 2 to 3 weeks.

The plasmid p35S/Ac (Hoechst Ag, Frankfurt, Germany) or equivalent may be used in transformation experiments in order to provide for a selectable marker. This plasmid contains the Pat gene (see, EP Patent Publication Number 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell, et al., (1985) Nature 313:810-812) and comprises the 3′ region of the nopaline synthase gene from the T-DNA of the Ti plasmid of Agrobacterium tumefaciens.

The particle bombardment method (Klein, et al., (1987) Nature 327:70-73) may be used to transfer genes to the callus culture cells. According to this method, gold particles (1 μm in diameter) are coated with DNA using the following technique. Ten pg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic™ PDS-1000/He biolistic particle delivery system (Bio-Rad Instruments, Hercules, Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains glufosinate (2 mg per liter) and lacks casein or proline. The tissue continues to grow slowly on this medium. After an additional 2 weeks the tissue can be transferred to fresh N6 medium containing glufosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839).

### Example 12

### Expression of Transqenes in Dicots

Soybean embryos are bombarded with a plasmid comprising a preferred promoter operably linked to a heterologous nucleotide sequence comprising a Bacterial MFS polynucleotide sequence or subsequence as follows. To induce somatic embryos, cotyledons of 3 to 5 mm in length are dissected from surface-sterilized, immature seeds of the soybean cultivar A2872, then cultured in the light or dark at 26° C. on an appropriate agar medium for six to ten weeks. Somatic embryos producing secondary embryos are then excised and placed into a suitable liquid medium. After repeated selection for clusters of somatic embryos that multiply as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are sub-cultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A DuPont Biolistic™ PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette of interest, comprising the preferred promoter and a heterologous Bacterial MFS polynucleotide, can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M) and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×5 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 13

### Field Trials under Nitrogen Stress and Normal Nitrogen Conditions

Corn hybrids containing a Bacterial MFS construct transgene are planted in the field under nitrogen-stress and normal-nitrogen conditions. Under normal nitrogen, a total of 250 lbs nitrogen is applied in the form of urea ammonium nitrate (UAN). Nitrogen stress is achieved through depletion of soil nitrogen reserves by planting corn with no added nitrogen for two years. Soil nitrate reserves are monitored to assess the level of depletion. To achieve the target level of stress, UAN is applied by fertigation or sidedress between V2 and VT growth stages, for a total of 50-150 lbs nitrogen.

Events from the construct are nested together with the null to minimize the spatial effects of field variation. Multiple reps are planted. The seed yield of events containing the transgene is compared to the yield of a transgenic null. Statistical analysis is conducted to assess whether there is a significant improvement in yield compared with the transgenic null, taking into account row and column spatial effects.

Differences in yield, yield components or other agronomic traits between transgenic and non-transgenic plants in reduced-nitrogen fertility plots may indicate improvement in nitrogen utilization efficiency contributed by expression of a transgenic event. Similar comparisons are made in plots supplemented with recommended nitrogen fertility rates. Effective transgenic events may achieve similar yields in the nitrogen-limited and normal nitrogen environments or may perform better than the non-transgenic counterpart in low-nitrogen environments.

In addition, the Bacterial MFS transgenic plants have increased sink capacity as result of mature ear length, mature ear width and kernel number per ear production. Realizing the yield potential may be achieved through increasing source strength and nutrient supply by either transgene manipulation or anronomic cultivation. Therefore, the Bacterial MFS transgenic may be used to increase yield under high N and fertilizer application, a condition most current commercial hybrids no longer respond to in yield increase, or plateau and are limited by sink capacity. Experiments where higher N levels per plant or higher photosynthetic activity per plants are created may demonstrate the value of combining Bacterial MFS with native germplasm, or other transgenic plants having more source production. The balance between sink size (kernel number/plant) and source size (photosynthetic carbon fixation) may be critical in securing commercial levels of improved yield.

### Example 14

### Evaluation of Construct for Effect on Yield Components

In order to measure the effect of transgene insertion on the yield components responsible for economic grain yield in maize, hybrid corn in grown under representative field conditions. The component values are measured in order to compare the plant results of the non-transformed plants, and/or wild type hybrids to the same hybrid containing the transgene insertion.

Plant seeds are planted in replicated field studies with common plant densities provided for all plots. Nutrient, water, insect control and weed control is provided to encourage good growth during the growing season. At maturity, measurements are performed on 10 sequential plants of the null and transgenic hybrids, including, but not limited to: number of ears, total number of kernels/plant, average weight per kernel. Calculations are performed to determine the total number of kernels produced/acre: kernels/plant×plants/acre, and uield (bu/acre): total kernels/acre X average weight/kernel. Constructs that improve one or more yield components, and/or calculated yield/acre would be deemed as having potential for improved commercial productivity in maize.

### Example 15

### Variant Sequences

Additional mutant sequences can be generated by known means including but not limited to truncations and point mutationa. These variants can be assessed for their impact on male fertility by using standard transformation, regeneration, and evaluation protocols.

A. Variant Nucleotide Sequences that do not Alter the Encoded Amino Acid Sequence

The disclosed nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants is altered, the amino acid sequence encoded by the open reading frames does not change. These variants are associated with component traits that determine biomass production and quality. The ones that show association are then used as markers to select for each component traits.

B. Variant Nucleotide Sequences in the Non-Coding Regions

The disclosed nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO. These variants are then associated with natural variation in the germplasm for component traits related to biomass production and quality. The associated variants are used as marker haplotypes to select for the desirable traits.

C. Variant Amino Acid Sequences of Disclosed Polypeptides

Variant amino acid sequences of the disclosed polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method. These variants are then associated with natural variation in the germplasm for component traits related to biomass production and quality. The associated variants are used as marker haplotypes to select for the desirable traits.

D. Additional Variant Amino Acid Sequences of Disclosed Polypeptides

In this example, artificial protein sequences are created having 80%, 85%, 90% and 95% identity relative to the reference protein sequence. This latter effort requires identifying conserved and variable regions from an alignment and then the judicious application of an amino acid substitutions table. These parts will be discussed in more detail below.

Largely, the determination of which amino acid sequences are altered is made based on the conserved regions among disclosed protein or among the other disclosed polypeptides.

Based on the sequence alignment, the various regions of the disclosed polypeptide that can likely be altered are represented in lower case letters, while the conserved regions are represented by capital letters. It is recognized that conservative substitutions can be made in the conserved regions below without altering function. In addition, one of skill will understand that functional variants of the disclosed sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 7.

TABLE 7
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the disclosed polypeptides are generating having about 80%, 85%, 90% and 95% amino acid identity to the starting unaltered ORF nucleotide sequence.

### Example 16

### Identification of new bacterial MFS Sequences

The 15 tested NRT sequences (SEQ ID NO: 4, 8, 11, 12, 19, 20, 22, 24, 32, 33, 35, 38, 42, 50 and 52) were used to search GenBank non-redundant database (NR) using the BLAST program. Protein sequences identified by Blast searches were filtered to include sequences that are within the 350 and 800 amino acids in length. Resulting sequences were clustered based on % sequence identity (% ID) and % query length overlap (% L). Clusters at 95′)/01D and 95% L were generated. The resulting sequences were aligned with ClustalW, default parameters and a phylogenetic tree was generated using Neighbor Joining algorithm in the JalView program.

Sequences adjacent to the 6 tested MFS sequences which showed ear traits efficacy at T0 generation and that belong to the same clade Genus/Family are claimed (Table 3 and 4).

### Example 17

### 3D Structure-Based Sequence Analysis and Modeling to Predict Gene Function of Bacterial MFS

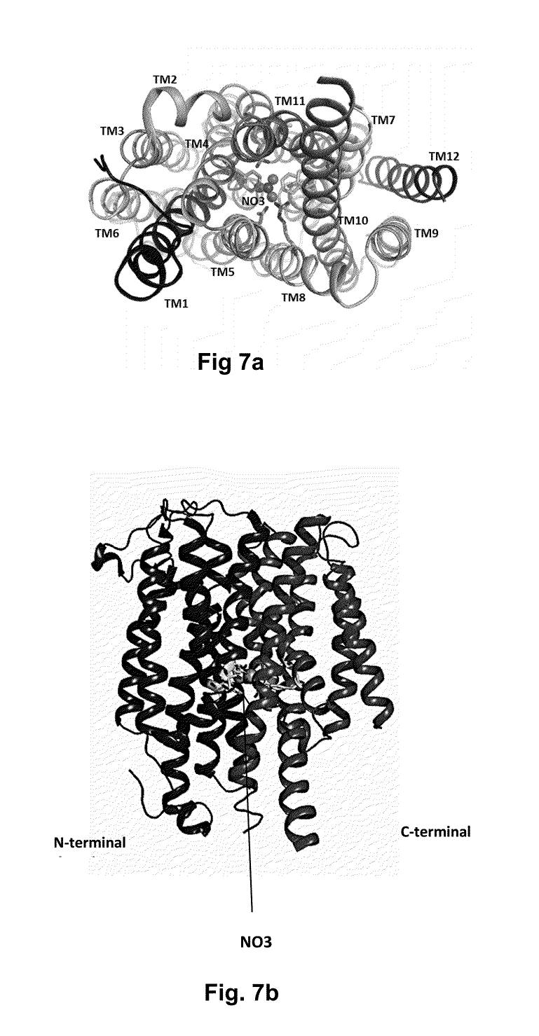

X-ray crystal structures of two nitrate/nitrite transporters, NarK and NarU from E. coli, have been solved to high resolution (Yan et. al. (2013) Cell Rep., 3:716-723; Zheng et al. (2013) Nature 497:547-651), providing excellent modeling templates for microbial proteins. Consistent with previous sequence analysis, both NarK and NarU structures sharing 76% sequence identity reveal not only the typical fold of MFS (major facilitator superfamily) but the specialized structural features for NNP (nitrate/nitrite porter) family and even substrate binding site characteristics unique to nitrate/nitrite transporters as well. NarK and NarU structures comprise of 12 transmembrane helices (TMs) which can be divided into two domains, TMs 1-6 and TMs 7-12 corresponding to N-terminal and C-terminal halves, respectively. Both domains are related by a pseudo 2-fold axis parallel to the membrane normal and each domain is able to rotate as a rigid body relatively one another, enabling a rocker-switch or alternatively access mechanism to transport substrates. Distinct from other MFS families, all the NNP family proteins uniquely contain two short Gly-rich sequence segments named as nitrate signature motifs NS1 and NS2. Both NS1 (GGALGLNGGLGN₁₇₅ in NarK) on TM5 and NS2 (GFISAIGAIGGFF₄₂₀ in NarK) on TM11 are located at the center of the transporter and line part of the substrate transport pathway. These abundant Glys enable the TM5 and TM11 to tightly fit the relative small substrate (nitrate/nitrite) and also give the central helices enough flexibility for conformational switch during transporting. Similar to other MFS structures, the nitrate binding site is at the center of N- and C-domain interface. The bound nitrate salt-bridges to two positively charged Args (R87 and R303 in NarU), hydrogen-bonds to two polar residues (N173 and Y261) in planar, and is finally sandwiched by F145 and F367 (FIG. 7). Mutagenesis study showed all these substrate binding residues are essential for transporter activity. In sharp contrast to other known MFS structures, both NarK and NarU's transport pathways are positively charged and lack protonatable residue (Glu, Asp, or His), indicating they might not be proton-driven transporter. More likely, NarK and NarU are nitrate/nitrite exchanger or use different gradients such as sodium/potassium for pumping.

To gain the structural perspective, a comparison of all the 110 microbial proteins to NarK sequence and mapped out all the essential structural features among the proteins with decent to high similarity was performed. Based on fasta36 match of Nark against the 110 microbial sequences, 71 of them can be aligned to NarK decently with E value less than 1e-4 (Table 8). Of them, Seq ID NO 51 (gi:89108312) from E. coli is best matched with >76% identity and its 3D structure is modeled with homology method. All the 71 sequences with NarK (PDB:4jre) and NarU (4iu8) are aligned with clustalw2 algorithm. Using NarK and NarU as reference, all the structure features on the multiple sequence alignment were marked out. Clearly, all the sequences except SEQ ID NO 35 have the non disrupted 12 TMs. The SEQ ID NO 35's TM1 is truncated by half from N-terminal. Considering TM1 is lumen lining helix, SEQ ID NO 35 must have modified function. Another 70 sequences all have conserved NS1 and NS2 nitrate signature and the nitrate binding residues except SEQ ID NO 177 in which the polar N (corresponding to N173 in NarU) is replaced by hydrophobic M. Thus this protein has a different substrate.

Further examination was performed on substrate binding sites (Table 8). In addition to conserved binding residues, SEQ ID NO 39, 105, 41, 43, and 47's substrate binding sites have two negatively charged Glus from TM7 and TM10 like some sugar transporters. Likewise, at the same position as TM10's Glu, SEQ ID NO 24, 25, 27, 28, 30, 31, 21, 23 have a positively charged Lys facing the lumen. These proteins likely have different substrate specificity.

SEQ ID NO 181, 182, and 12 have a protonatable Glu on TM1 pointing to the lumen, making pH-driven transport possible.

Twenty-eight sequences (SEQ ID NO 13, 14, 15, 16, 18, 180, 17, 19, 126, 121, 122, 124, 125, 123, 108, 109, 107, 110, 111, 118, 120, 116, 113, 112, 117, 119, 115, and 20) have Asp in middle of TM1 facing the hydrophobic bilayer. Half of them (SEQ ID NO 108, 109, 107, 110, 111, 118, 120, 116, 113, 112, 117, 119, 115, and 20) also have Asp on TM9. As the charged residues are often prohibited to contact membrane core, the local helical conformation is likely disrupted. After a quarter turn twist of helical phase, these Asps could face to the lumen, likely altering the substrate and driven force specificity.

The remainder of the 25 sequences (SEQ ID NO 34, 33, 7, 9, 8, 5, 6, 2, 3, 1, 131, 132, 130, 129, 128, 4, 135, 133, 134, 127, 51, 183, 184, 10, and 11) have all the critical features of NarK/NarU and also lack the protonatble residues. They are more likely to function as NarK/NarU based on the structural analysis.

While the foregoing subject matter has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications and/or other documents cited in this application are incorporated by reference in their entirety for all purposes to the same extent as if each individual publication, patent, patent application and/or other document were individually indicated to be incorporated by reference for all purposes.

What is claimed is:

3. An isolated polynucleotide selected from the group comprising:
a. a polynucleotide encoding a polypeptide selected from the group consisting of SEQ ID NOS: 1, 2 and 5-32;
b. a polynucleotide selected from the group consisting of SEQ ID NOS: 53-104 or 136-166; and
c. a polynucleotide having 85% sequence identity to SEQ ID NOS: 53-104 or 136-166,
operably linked to a regulatory element that functions in plants.

4. The isolated nucleic acid of claim 3 wherein said regulatory element is a constitutive promoter.

5. The isolated nucleic acid of claim 3, wherein expression of the nucleic acid results in the expression of one or more bacterial MFS (major facilitator superfamily) genes in a plant cell.

6. A plant or plant cell comprising the isolated nucleic acid of claim 3.

8. A plant or plant cell comprising an expression cassette effective for expression of at least one bacterial MFS gene, wherein said expression cassette comprises a promoter that functions in plants operably linked to a nucleic acid, wherein said nucleic acid comprises polynucleotides of claim 3.

9. The plant cell of claim 8, wherein the plant cell is from a dicot or monocot.

11. A plant regenerated from the plant cell of claim 9.

12. The plant of claim 6, wherein the plant exhibits one or more of the following: increased drought tolerance, increased nitrogen utilization efficiency, increased seed yield, increased biomass yield, increased density tolerance and increased density tolerance, compared to a control plant.

13. A method of increasing sink capacity and/or grain dry down in a plant, the method comprising reducing the expression of one or more bacterial MFS genes in the plant, by expressing a transgenic nucleic acid comprising a nucleotide sequence selected from the group consisting of claim 3.

14. The method of claim 13, wherein the transformed plant exhibits one or more of the following: (a) an increase in the production of at least one bacterial MFS; (b) an increase in the production of a bacterial MFS protein; (c) a increase in sink capacity; (d) an increase in ear number and or kernel number; (e) an increase in drought tolerance; (f) an increase in nitrogen utilization efficiency; (g) an increase in density tolerance; (h) an increase in plant height or (i) any combination of (a)-(h), compared to a control plant.

15. A method of increasing yield or drought tolerance in a plant, the method comprising increasing the expression of one or more bacterial MFS genes in the plant by expressing the nucleic acid of claim 3.

16. A method of increasing drought tolerance in the absence of a yield penalty under non-drought conditions, the method comprising increasing the activity of one or more nucleic acid sequences encoding a polypeptide claim 3.

18. Seed of the plant of claim 8, wherein the seed comprises the expression cassette.

19. The method of increasing source capacity of the bacterial MFS transgenic plants to support the increased sink capacity in order to realize increased yield potential.

20. The method of claim 19, where the increased yield potential is due to mature ear length, mature ear width and kernel number per ear.

21. The method of claim 19, which includes increasing source strength of the bacterial MFS transgenic plants by stacking with other genes for more biomass production, photosynthesis or any forms of the transgene manipulation.

22. The method of claim 19, which includes increasing soil fertility through N and fertilizer applications to improve source strength.

23. The method of claim 15, further comprising increasing stalk strength.

24. The method of claim 15, further comprising increasing the availability of nitrogen for enhanced sink capacity.

25. A method of increasing the expression of bacterial MFS or the activity of bacterial MFS in a plant, the method comprising modulating the expression levels of bacterial MFS or the protein level of bacterial MFS or the activity of bacterial MFS polypeptide, wherein the modulation results in an improved agronomic performance of the plant.
